(19) United States
(12) Patent Application Publication
(10) Pub. No.: US 2013/0209368 A1
(43) Pub. Date: Aug. 15, 2013

(54) NEAR INFRARED FLUORESCENT PARTICLES AND USES THEREOF

(71) Applicants: Shlomo Magdassi, Jerusalem (IL); Philip Lazarovici, Jerusalem (IL); Liraz Larush, Jerusalem (IL); Emma Portnoy, Beer-Sheva (IL); Shimon Lecht, Petach Tikva (IL); Ran Tivony, Jerusalem (IL)

(72) Inventors: Shlomo Magdassi, Jerusalem (IL); Philip Lazarovici, Jerusalem (IL); Liraz Larush, Jerusalem (IL); Emma Portnoy, Beer-Sheva (IL); Shimon Lecht, Petach Tikva (IL); Ran Tivony, Jerusalem (IL)

(73) Assignee: YISSUM RESEARCH DEVELOPMENT COMPANY OF THE HEBREW UNIVERSITY OF JERUSALEM LTD, Jerusalem (IL)

(21) Appl. No.: 13/822,095

(22) PCT Filed: Sep. 8, 2011
(86) PCT No.: PCT/IL2011/000725

§ 371 Date: May 1, 2013

Related U.S. Application Data
(60) Provisional application No. 61/381,190, filed on Sep. 9, 2010.

(51) Int. Cl. A61K 49/00

(52) U.S. Cl. CPC A61K 49/0084
USPC 424/9.6; 424/178.1; 514/9.6; 427/2.11

(57) ABSTRACT

The present invention provides particles comprising either a water-soluble polymer or a phospholipid, wherein at least one near infrared (NIR) fluorescent probe and optionally at least one active agent such as a targeting moiety, capable of selectively recognizing a particular cellular marker, are non-covalently bound to the outer surface of the particles. Pharmaceutical compositions comprising these particles may be used, inter alia, for detection and treatment of tumors in the gastrointestinal tract

### TECHNICAL FIELD

The present invention provides particles comprising either a water-soluble polymer or a phospholipid, wherein at least one near infrared (NIR) fluorescent probe is non-covalently bound to the outer surface of said particle, as well as pharmaceutical compositions and uses thereof.

### BACKGROUND ART

Indocyanine green (ICG) is a water-soluble tricarbocyanine dye that absorbs and emits in the near infrared (NIR) range. It is the only US Food and Drug Administration (FDA)-approved NIR molecule that has over 50 years of clinical experience with an excellent safety record, which makes it very attractive for various in vivo applications (Dzurinko et al., 2004; Sakka, 2007).

In current clinical setups, ICG is used in aqueous solution where it has a relatively low quantum yield (Philip et al., 1996; Sevick-Muraca et al., 1997), and undergoes rapid chemical degradation and aggregation, which causes decreased absorption and fluorescence (Saxena et al., 2003). Recently, cumulative data indicate that immobilization of ICG onto various surfaces may result in improved stability and therefore improved fluorescence properties (Desmettre et al., 2000; Gathje et al., 1970). Such immobilization was obtained by embedding the ICG molecule within polymeric nanoparticles (Saxena et al., 2004; Yaseen et al., 2009; Yu et al., 2010) and by inclusion in liposomes (Devoiselle et al., 1997; Proulx et al., 2010; Sandanaraj et al., 2010) and micelles (Devoiselle et al., 1997; Kirchherr et al., 2009).

Liposomes are a very attractive delivery form because they are physically and chemically well-characterized structures that can be delivered through almost all routes of administration, and are biocompatible (Barenholz and Crommelin, 1994). Utilization of ICG-loaded liposomes in biological systems was recently described by Sandanaraj et al. (2010) and Proulx et al. (2010).

Carcinomas are common but difficult-to-treat diseases. Surgery, chemotherapy, and radiation are common treatment; however, do not always cure the disease. Thus, improvement of tumor diagnosis will assist in the follow-up of therapeutic regimens, based on defined biomarkers (Duffy et al., 2007).

Colonic polyps are slow-growing overgrowths of the colonic mucosa, highly prevalent in the general population, especially with increasing age. While approximately 90% of the polyps are less than 1 cm in diameter and have a low potential for malignancy, the remaining 10% of adenomas are larger than 1 cm in diameter, and those of which containing a substantial (>25%) villous component or having high-grade dysplasia are commonly referred to as advanced neoplasms and carry an increased cancer risk (Enders, 2009).

Colorectal cancer (CRC) screening is aimed at reducing CRC mortality through the identification of advanced neoplasia, wherein effective CRC prevention has been closely related to the detection and removal of advanced adenomas. Although colonoscopy is the acknowledged gold standard for detecting colon neoplasms (Ferrucci, 2003), a recent study found a poor detection rate of 48% for polyps of 10 mm in diameter or more, as well as a poor overall detection rate of 39% only for adenomas. Obviously, specific marking of the lesion by optical probes may significantly improve the detection and therefore reduce morbidity and mortality from cancer.

The tyrosin kinases, representing a family of about 60 physiological proteins and their oncogenic forms, are characterized by an extracellular ligand-binding domain, a transmembrane region, and a tyrosine kinase catalytic motif in the intracellular part. A typical family of these receptors is represented by the epidermal growth factor receptor (EGFR) family, which is overexpressed in many carcinomas such as those in breast, prostate and gastrointestinal organs. Preclinical and clinical studies have shown that targeting the EGFR family is a valid strategy for diagnosis and anticancer therapy. In preclinical studies, for diagnostic purposes, EGFR-expressing tumors both in vitro and in vivo were imaged using a variety of approaches such as EGFR-targeted immunoliposomes with the aid of selective antireceptor monoclonal antibodies and fragments; fluorescent labeling of FDA-approved monoclonal antibodies such as cetuximab (Erbitux®), trastuzumab (Herceptin®), and daklizumab (Zenepax); quantum dots conjugated with either EGF or monoclonal antibodies to the EGFR family; and ¹¹¹In-labeled human EGF (Becker et al., 2006; Flu et al., 2007; Koyama et al., 2007; Kriete et al., 2006; Mamot et al., 2006; Tada et al., 2007). Recently, NIR-labeled EGF was prepared and demonstrated to be a useful tool to study EGFR-overexpressing tumors (Gong et al., 2010). This approach utilizes the natural ligand of EGFR, which may cause receptor activation and further amplify the mutagenic activity of the tumor; therefore, to circumvent this possibility the FDA-approved monoclonal antibody for EGFR may be used as a targeting moiety, lacking the receptor activation ability.

The Thomsen-Fiedenreich (TF) antigen is specifically expressed on the mucosal side of cancer cells in the early stage of colorectal cancer. In addition, increased expression of TF antigen has been demonstrated in hyperplastic and adenomatous colonic polyps, and in inflammatory bowel disease. Its terminal sugar is galactose β-D-galactasyl-(1-3)-N-acetyl-D-galactosamine (Gal-(1-3)GalNAc), and it is masked by an oligosaccharide side chain extension or sialylation in normal cells. It is known that peanut (Arachis hypogaea) agglutinin (PNA) binds specifically to the TF antigen through the recognition of Gal-(1-3)GalNAc. During tumorigenesis, an aberrant glycosylation takes place in Golgi apparatus leading to over secretion of TF antigen into the cytoplasm along with mucin granules and later into cell membrane (Singh et al., 2001; Dabelsteen, 1996; Campbell et al., 1995; Boland et al., 1988). PNA can be utilized for ex vivo and potentially in vivo diagnosis as previously shown (Sakuma et al., 2009).

Nanoparticles possess enormous potential as diagnostic imaging agents and hold promise for the development of multi-modality agents with both imaging and therapeutic capabilities. Moreover, particles with targeting and biological recognition towards selective biomarkers expressed by carcinoma tumors will provide novel diagnostic tools. The creation of biocompatible nanoparticles conjugated with antibody or EGF ligand and adsorbed by NIR fluorescent dyes will provide an advanced enabling technology for diagnosis of tyrosine kinase receptor family in general and EGFR in particular.

Lately, there is increasing interest in the use of nanoparticles containing ICG probe and having a specific recognition to a targeted organ, system or tumor for in vitro and in vivo imaging. Particular publications disclose, e.g., ICG injectable solution for checking the accuracy of cerebral blood flow measurements (Leung et al., 2007) or for measuring blood flow in the retinal surface and sub retinal space of rabbit eyes (Maia et al., 2004). WO 2006/076636 discloses a composition comprising colloids stable under physiological pH and ionic strength, and comprising particles having a core and a shell, wherein (i) the shell comprises a polymer having amine functionalities; (ii) the particles have a volume-weighted mean particle size diameter of less than 200 nm; and (iii) more than 50% of the polymer in the colloid is bound to the core surfaces. WO 2007/025768 discloses a nanoparticle matrix comprising a co-aggregate of at least one charged polyelectrolyte and at least one oppositely charged active agent, wherein the active agent is a hydrophilic optically fluorescent agent.

Other studies describe the effect of the solvent medium and the dye-containing nanoparticles' shape on the fluorescence efficiency (Horimoto et al., 2008), or conditions for coupling a biomolecule or loading of cells with ICG above its quenching concentration. As reported, after cell binding and internalization into a target organ, ICG dissociates from the carrier thus reaching a concentration which enables to emit photons (Ogawa et al., 2009).

### SUMMARY OF INVENTION

In one aspect, the present invention provides a particle comprising a water-insoluble polymer, wherein at least one near infrared (NIR) fluorescent probe is non-covalently hound to the outer surface of said particle. In certain embodiments, the particle of the invention further comprises at least one active agent non-covalently bound to the outer surface of said particle.

In another aspect, the present invention provides a particle comprising a phospholipid, wherein at least one NIR fluorescent probe and at least one active agent are non-covalently bound to the outer surface of said particle.

In a further aspect, the present invention provides a powder comprising freeze dried- or spray dried-particles as defined above, i.e., either water-insoluble polymer based- or phospholipid based-particles.

In still another aspect, the present invention provides a pharmaceutical composition comprising particles as defined above, i.e., either water-insoluble polymer-based- or phospholipid-based-particles, or a powder as defined above, and a pharmaceutically acceptable carrier. In certain embodiments, the pharmaceutical composition of the invention is formulated for oral administration. Such compositions are particularly useful in detection and/or treatment of a gastrointestinal cancer or a pre-cancer state in the gastrointestinal tract.

In still a further aspect, the present invention thus relates to a method for detection of a cancer or a pre-cancer state in the gastrointestinal tract of an individual in need, said method comprising administering to said individual a pharmaceutical composition as defined above, formulated for oral administration, and detecting the presence of NIR emission from the walls of the gastrointestinal tract upon excitation at a proper wavelength.

In yet a further aspect, the present invention relates to a method for treatment of a cancer or a pre-cancer state in the gastrointestinal tract of an individual in need, said method comprising administering to said individual a therapeutically effective amount of a pharmaceutical composition as defined above, formulated for oral administration.

In another aspect, the present invention relates to a method for the preparation of an aqueous dispersion of particles comprising a water-insoluble polymer, to the outer surface of which at least one NIR fluorescent probe is non-covalently bound, said method comprising the steps of:
(i) dissolving a water-insoluble polymer in an organic solvent to obtain an organic solution of said polymer;
(ii) mixing said organic solution with an aqueous solvent in which a surfactant is optionally dissolved, in a ratio of 40:60 to 5:95, respectively, and removing said organic solvent to obtain an aqueous dispersion comprising particles of said polymer;
(iii) optionally mixing the aqueous dispersion of (ii) with an aqueous solution of a protein, peptide or polypeptide, to obtain an aqueous dispersion comprising particles of said polymer to the outer surface of which said protein, peptide or polypeptide is adsorbed;
(iv) mixing the aqueous dispersion of (ii) or (iii) with an aqueous solution of at least one NIR fluorescent probe thereby non-covalently binding said at least one fluorescent probe to the outer surface of said particles; and
(v) removing unbound molecules of said fluorescent probe from the dispersion obtained in (v).

In a further aspect, the present invention relates to a method for the preparation of an aqueous dispersion of particles comprising a phospholipid, to the outer surface of which at least one NIR fluorescent probe and at least one active agent are non-covalently bound, said method comprising the steps of:
(i) Providing an aqueous dispersion of particles comprising a phospholipid;
(ii) mixing the aqueous dispersion of (i) with an aqueous solution of at least one NIR fluorescent probe thereby non-covalently binding said at least one fluorescent probe to the outer surface of said particles;
(iii) removing unbound molecules of said fluorescent probe from the dispersion obtained in (ii);
(iv) mixing the aqueous dispersion of (iii) with an aqueous solution of said at least one active agent thereby non-covalently binding said at least one active agent to the outer surface of said particles; and
(v) removing unbound molecules of said at least one active agent from the dispersion obtained in (iv).

### BRIEF DESCRIPTION OF DRAWINGS

FIG. 1 shows cryo-TEM image of liposomes labeled by 3.2×10⁻⁵ M indocyanine green (ICG).

FIGS. 2A-2B show adsorption isotherm of ICG bound to liposomes as a function of Free ICG at equilibrium (2A); and zeta potential of liposome dispersion as function of ICG concentration (2B).

FIGS. 3A-3B show fluorescence of ICG in buffer (⋄) and ICG-adsorbed liposome dispersion at various ICG concentrations ( [symbol] ). Excitation at 720 nm; the emission intensities are presented at the wavelengths in which the peak was observed (3A); and effect of ICG concentration in liposomal dispersion on fluorescence shift (▪) and fluorescence intensity () (3B).

FIG. 4 shows that while dissolved ICG absorption decreases over time both in light and in dark, the absorption of ICG when bound to the liposomes almost does not change, and that while light further decreases dissolved ICG absorption due to photobleaching, the absorption of ICG when bound to the liposomes remains the same.

FIG. 5 shows the stability of ICG-adsorbed liposomes in human colon fluid. Images were taken by Odyssey MR laser scanner (Ex 785 nm and Em 800 nm).

FIG. 6 shows IgG binding presented as TMB absorbance (HRP, substrate) as a function of liposome concentration. IgG concentration was kept constant throughout this experiment.

FIGS. 7A-7D show purification and bioactivity validation of EGF-FITC. Purification of FITC-labeled EGF was performed in two sequential steps of desalting (7A) and ion exchange (7B). A431 cultures were incubated with 10 ng/ml for 10 min with native (EGF) and FITC-labeled EGF (EGF-FITC) or left untreated. The phosphorylation of Erk (p-Erk) was evaluated using Western blotting. Total Erk protein (pan-Erk) was measured for internal control purposes to assure loading of equal protein quantities (7C). A431 cells were incubated for 15 min with 10 ng/ml EGF-FITC in the presence or absence of excess unlabeled EGF (500 ng/ml) or left untreated. The cultures were visualized using fluorescent microscopy at 40× magnification (7D).

FIG. 8 shows binding of fluorescein-containing liposomes to A431 carcinoma cell culture visualized by confocal microscopy. The cultures were incubated for 40 min at 37° C. with 10 ng/ml of nonlabeled liposomes (panel A), EGF-FITC (panel B), IgG-labeled liposomes (Lipo-FITC-IgG) (panel C), IgG-labeled liposomes (panel C′), cetuximab-labeled liposomes (Lipo-FITC-Cetuximab) (panel D), and cetuximab-labeled liposomes (panel D′). Scale bar, 25 μm. Panel E shows fluorescent intensities at cell membrane (white bars) or cytosol (black bars) quantified and presented in arbitrary units (a.u.). #p<0.05 vs. membrane IgG-FITC-labeled-liposomes, *p<0.05 vs. cytosol IgG-FITC-labeled liposomes.

FIG. 9 shows NIR imaging of living cells: binding of ICG-containing liposomes to A431 carcinoma cell culture in the center and IEC-6 normal colon cells in the surrounding. The cultures were incubated for 30 min at 37° C. with 10 ng/ml of various liposome preparations. Lipo—liposomes only; Lipo-IgG—control liposomes containing IgG as targeting moiety; Lipo-Cetux—liposomes containing cetuximab as targeting agent. Upper panel shows representative images of the in vitro focal colon cancer model. * p<0.05 vs. Lipo.

FIG. 10 shows the calculated quantity of PNA-FITC adsorbed to ICG-adsorbed liposomes as a function of PNA concentration, using fluorescence.

FIG. 11 shows PNA-FITC-labeled ICG-adsorbed liposomes applied to DMH-treated rat colon. The upper panel is a photograph showing polyps (marked in red) in the rat colon, and the lower panel is an image by Odyssey NIR scanner system after 30 min of incubation with PNA-FITC-labeled ICG-adsorbed liposomes and washing.

FIG. 12 shows scanning electron microscope image of Eudragit-RS nanoparticles.

FIG. 13 shows adsorption isotherm of ICG to Eudragit-RS nanoparticles as a function of free ICG concentration.

FIGS. 14A-14B show fluorescence and absorbance of ICG solution and ICG-adsorbed Eudragit RS nanoparticles. ICG aqueous solution and nanoparticles loaded with ICG fluorescent intensity as a function of ICG concentration (14A); and absorption and emission profile of ICG-loaded nanoparticles compared with ICG aqueous solution both at a concentration of 10⁻⁶ M (14B).

FIG. 15 shows imaging of fluorescence by a NIR laser scanner of: test tubes containing a dispersion of ICG-loaded Eudragit RS nanoparticles, and ICG solution (right), and the same tubes under 1.6 cm thickness tissue mimicking solid (left).

FIG. 16 shows fluorescence intensity of both dyes ICG and fluorescein isothiocyanate (FITC), obtained from Euidragit RS 100 nanoparticles loaded with ICG binding to FITC human IgG. Em: Emission; Ex: Excitation.

FIG. 17 shows IgG adsorption to the Eudragit RS 100 nanoparticles as a function of IgG concentration using sandwich ELISA.

FIG. 18 shows stability test of ICG-loaded Eudragit RS 100 nanoparticles binding to fluorescein isothiocyanate human IgG in intestinal fluid, pH 6.8, at 37° C. up to 8 hrs.

FIG. 19 shows EGF binding to ICG-adsorbed polycation nanoparticles as a function of EGF concentration, using ELISA.

FIG. 20 shows AFM image of Eudragit-casein nanoparticles prepared as described in Example 16 (panel B shows a magnification of panel A). The size values (horizontal, vertical and surface distance, respectively) measured for the three nanoparticles indicated in panel B are 0.204 μm, 1.771 nm and 0.224 μm (nanoparticle 1); 0.088 μm, −0.380 nm and 0.094 μm (nanoparticle 2); and 0.142 μm, 0.506 nm and 0.159 μm (nanoparticle 3).

FIG. 21 shows stability of ICG-adsorbed Eudragit-casein nanoparticles in human colon fluid. Images were taken by Odyssey NIR laser scanner (Ex 785 nm and Em 800 nm).

FIG. 22 shows cetuximab binding to ICG-adsorbed Eudragit-casein nanoparticles as a function of cetuximab concentration, using ELISA.

FIG. 23 shows PNA binding to ICG-adsorbed Eudragit-casein nanoparticles as a function of PNA concentration, using fluorescence.

FIG. 24 shows anti-CEA binding to ICG-adsorbed Eudragit-casein nanoparticles as a function of anti-CEA-FITC concentration by fluorescence measurement.

FIG. 25 shows in vivo specific recognition in LS174T tumor bearing mice by ICG-adsorbed Eudragit-casein nanoparticles without (upper panels) and with (lower panels) anti-CEA: left: light image of the mouse colon; middle: image processing—fluorescence intensity by natural basis (2.7); and right: Odyssey NIR laser scanner image (Ex 785 nm and Em 800 nm).

### DETAILED DESCRIPTION OF THE INVENTION

The present invention provides biocompatible, stable micro- and nano-particles that are fluorescent in the near infrared (NIR) range and capable of specific bio-recognition and targeting. The term “biocompatible” as used herein means that the particles of the invention are made of compounds suitable for administration, including oral administration, to humans; and the term “stable” as used herein means that these particles are both physically and chemically stable, and are neither degraded nor aggregated under physiological conditions including in intestinal and colon fluids.

In one particular aspect, the present invention provides a particle comprising a water-insoluble polymer, wherein at least one near infrared (NIR) fluorescent probe is non-covalently bound, i.e., adsorbed, to the outer surface of said particle.

In certain embodiments, the particle of the present invention comprises a water-insoluble cationic polymer. In particular embodiments, the cationic polymer is selected from acrylic copolymers, methacrylic copolymers or acrylic-methacrylic copolymers. Examples of such copolymers include, without being limited to, Eudragit® RS, Eudragit® RS 30D, Eudragit® RL 30D, Eudragit® RL 100 and Eudragit® RL PO, wherein Eudragit® RS is preferred. In other particular such embodiments, the cationic polymer is chitosan.

In certain particular embodiments, the water-insoluble cationic polymer comprised within the particle of the invention is admixed with a suitable surfactant. Examples of surfactants that may be used according to the invention include, without limiting, nonionic surfactants such as polysorbates, e.g., Polysorbate 80 (Tween 80) and Polysorbate 20 (Tween 20); hydrogenated castor oil such as Polyoxyl 40 hydrogenated castor oil (Cremophor® RH 40), Polyoxyl 60 hydrogenated castor oil (Cremophor® RH 60) and Polyoxyl 35 castor oil (Cremophor® EL); Pluronic block copolymers such as Pluronic® L81, Pluronic® P85, Pluronic® F68, Pluronic® F127 and Pluronic® L44 NF INH; Lutrol® F127, Solutol® HS-15 or d-α-tocopheryl polyethylene glycol 1000 succinate (TPGS), and ionic surfactants such as lecithin and bile salts.

The cationic polymer-based particles of the invention are positively charged and have zeta potential of ≧5 mV. Nevertheless, since such particles may have non specific binding to healthy cells, their zeta potential may be changed or reversed by adsorbing a protein, peptide or polypeptide to the outer surface of the particles, thus neutralizing positive charges on the surface of the particles. The term “protein”, as used with this respect, refers to any protein having negatively charged groups as well as to proteinates such as those formed by dissolving a protein in a metal hydroxide. Particular examples of proteins that can be used so as to neutralize positive charges on the surface of the cationic polymer-based particles of the invention include, without being limited to, casein such as α-casein and β-casein, as well as caseinates such as sodium caseinate, calcium caseinate and magnesium caseinate, human serum albumin, bovine serum albumin, whey protein, β-lactoglobulin, α-lactalbumin, ovalbumin, lysozyme, a soy protein such as soy sodium proteinate and soy 11S, or wheat protein. Cationic polymer-based particles according to the invention, in which a protein, peptide or polypeptide is adsorbed to their outer surface so as to neutralize positive charges, are negatively charged and have zeta potential of ≧|10| mV (absolute value).

The NIR fluorescent probe non-covalently hound to the outer surface of the polymer-based particles of the invention may be any fluorescent probe having an absorption and fluorescence spectrum in the NIR region. Examples of such fluorescent probes include, without being limited to, cyanine dyes such as indocyanine green (ICG), Cy5, Cy5.5, Cy5.18, Cy7 and Cy7.18; IRDye 78, IRDye 680, IRDye 750, IRDye 800 phosphoramidite (LI-COR Biosciences), DY-681, DY-731, DY-781 (Dyomics GmbH), or Alexa Fluor dyes such as Alexa Fluor® 610, Alexa Fluor® 633, Alexa Fluor® 647, Alexa Fluor® 660, Alexa Fluor® 680, Alexa Fluor® 700 and Alexa Fluor® 750. In a particular embodiment, the NIR fluorescent probe non-covalently bound to the outer surface of the particle of the invention is ICG, which is the only US FDA-approved NIR molecule. In one embodiment, the polymer-based particle of the invention comprises less than 0.005% by weight of said NIR fluorescent probe.

In certain embodiments, the polymer-based particle of the invention further comprises at least one active agent non-covalently bound, i.e., adsorbed, to the outer surface of said particle.

According to the present invention, the active agent non-covalently bound to the outer surface of the polymer-based particles of the invention each independently may be selected from a peptide, a polypeptide, a protein such as an antibody, a lectin or a ligand-binding fragment thereof, a hormone or an analogue thereof, a glycoprotein, a lipoprotein, an amino acid, a polysaccharide, a glycolipid, a vitamin, a cofactor, a nucleoside, a nucleotide, or a nucleic acid such as RNA and DNA.

In certain embodiments, the active agent adsorbed to the outer surface of the particles of the invention is, in fact, a targeting moiety capable of selectively recognizing a particular cellular marker of a desired target thus binding the particles to said target. In particular such embodiments, the target to be recognized by the targeting moiety adsorbed to the outer surface of the particles is a tumor such as a gastrointestinal cancer, or a pre-cancer state, i.e., cells undergoing pre-cancer transformation, such as a colonic polyp or adenoma, i.e., a benign epithelial neoplasm (an abnormal new growth of tissue) that arises from the epithelial cells lining the colon; and the targeting moiety is capable of binding a specific cellular marker of said tumor or pre-cancer state such as a cell surface receptor or a cell surface glycoprotein. In other particular such embodiments, the active agent adsorbed to the outer surface of the particles of the invention is an anti-epidermal growth factor receptor (EGFR) antibody such as cetuximab (a chimeric [mouse/human] monoclonal antibody that is an EGFR inhibitor, marketed under the name Erbitux®, given by intravenous infusion for treatment of, e.g., metastatic colorectal cancer), an epidermal growth factor (EGF), or a lectin capable of binding galactosyl β-1,3-N-acetyl galactosamine (Gal β-1,3-GalNAc; Thomsen Friedenreich antigen) such as peanut agglutinin (Arachis hypogaea lectin).

In certain embodiments, the polymer-based particle of the invention comprises at least one NIR fluorescent probe and an active agent as defined above, both non-covalently bound to the outer surface of the particle, and one or more, e.g., two, three or four, additional active agents each independently non-covalently bound to the outer surface of the particle or embedded to the particle. In particular embodiments, the active agent non-covalently bound to the outer surface of the particle is a targeting moiety capable of selectively recognizing a particular cellular marker of a desired target, thus binding the particles to said target, and a further active agent, e.g., a therapeutic agent such as a chemotherapeutic agent, is either non-covalently bound to the outer surface of the particle or non-covalently embedded to the particle.

In another particular aspect, the present invention provides a particle comprising a phospholipid, wherein at least one NIR fluorescent probe and at least one active agent are non-covalently bound, i.e., adsorbed, to the outer surface of said particle.

In certain embodiments, the phospholipid comprised within the particle of the invention is selected from a lecithin such as egg or soybean lecithin; a phosphatidylcholine such as egg phosphatidylcholin; a hydrogenated phosphatidylcholine; a lysophosphatidylcholine; dipalmitoylphosphatidylcholine; distearoylphosphatidylcholine; dimyristoylphosphatidylcholine; dilauroylphosphatidylcholine; a glycerophospholipid such as phosphatidylglycerol, phosphatidylserine, phosphatidylethanolamine, lysophosphatidylethanolamine, phosphatidylinositol, phosphatidylinositol phosphate, phosphatidylinositol bisphosphate and phosphatidylinositol triphosphate; sphingomyelin; cardiolipin; a phosphatidic acid; a glycolipid such as a glyceroglycolipid, e.g., a galactolipid and a sulfolipid, a glycosphingolipid, e.g., a cerebroside (a glucocerebroside and a galactocerebroside), and a glycosylphosphatidylinositol; a plasmalogen; a phosphosphingolipid such as a ceramide phosphorylcholine, a ceramide phosphorylethanolamine and a ceramide phosphorylglycerol; or a mixture thereof.

In certain embodiments, the phospholipid comprised within the particle of the invention is admixed with one or more nonphosphorous-containing molecules. Non-limiting examples of suitable nonphosphorous-containing molecules include fatty amines such as octylamine, laurylamine, N-tetradecylamine, hexadecylamine, stearylamine, oleylamine, tallowamine, hydrogenated tallowamine, and cocoamine; fatty acids; fatty acid amides; esters of fatty acid such as isopropyl myristate, hexadecyl stearate, and cetyl palmitate; cholesterol; cholesterol esters; diacylglycerols; or glycerol esters such as glycerol ricinoleate.

Due to their size (≦100 nm) and zeta potential, the phospholipid-based particles more effectively penetrate mucus barrier. This fact is very encouraging since conventional particles, when administered to mucosal tissues, are likely to be trapped by the mucus and rapidly eliminated via the mucus clearance. Particles in size of less than 100 nm fit the mucus mean pore size and are therefore capable of penetrating the mucus. In order to further aid these particles to penetrate the mucus, a polyethylene glycol (PEG)ylated phospholipid can be admixed with the phospholipid composing the particle so as to minimize the interactions between the mucus and the particle (Lai et al., 2009).

In certain embodiments, the phospholipid comprised within the particle of the invention is thus admixed with one or more PEGylated phospholipids. Examples of PEGylated phospholipids that can be used according to the present invention include, without being limited to, PEGylated dipalmitoyl phosphatidylethanolamine (DPPE-PEG), PEGylated palmitoyloleoyl phosphatidylethanolamine (POPE-PEG), PEGylated dioleoyl phosphatidylethanolamine (DOPE-PEG) and PEGylated distearoyl phosphatidylethanolamine (DSPE-PEG), preferably 1,2-distearoyl-sn-glycero-3-phosphoethanolamine-N-[polyethyleneglycol 2000] (PEG-DSPE-2000). In a particular embodiment, the particle of the present invention comprises a phospholipid, optionally admixed with one or more nonphosphorous-containing molecules as defined above, and further admixed with PEG-DSPE-2000, wherein the particle comprises up to 15% by weight of PEG-DSPE-2000.

Phospholipid-based particles according to the invention are negatively charged and have zeta potential of ≧|10| mV (absolute value).

The NIR fluorescent probe non-covalently bound to the outer surface of the phospholipid-based particles of the invention may be any fluorescent probe having an absorption and fluorescence spectrum in the NIR region. Examples of such fluorescent probes include, without being limited to, cyanine dyes such as ICG, Cy5, Cy5.5, Cy5.18, Cy7 and Cy7.18; IRDye 78, IRDye 680, IRDye 750, IRDye 800 phosphoramidite (LI-COR Biosciences), DY-681, DY-731, DY-781 (Dyomics GmbH), or Alexa Fluor dyes such as Alexa Fluor® 610, Alexa Fluor® 633, Alexa Fluor® 647, Alexa Fluor® 660, Alexa Fluor® 680, Alexa Fluor® 700 and Alexa Fluor® 750. In a particular embodiment, the NIR fluorescent probe non-covalently bound to the outer surface of the particle of the invention is ICG. In one embodiment, the phospholipid-based particle of the invention comprises less than 0.005% by weight of said NIR fluorescent probe.

The active agent non-covalently bound to the outer surface of the phospholipid-based particles of the invention each independently may be selected from a peptide, a polypeptide, a protein such as an antibody, a lectin or a ligand-binding fragment thereof, a hormone or an analogue thereof, a glycoprotein, a lipoprotein, an amino acid, a polysaccharide, a glycolipid, a vitamin, a cofactor, a nucleoside, a nucleotide, or a nucleic acid such as RNA and DNA.

In certain embodiments, the active agent adsorbed to the outer surface of the phospholipid-based particles of the invention is, in fact, a targeting moiety capable of selectively recognizing a particular cellular marker of a desired target thus binding the particles to said target. In particular such embodiments, the target to be recognized by the targeting moiety adsorbed to the outer surface of the particles is a tumor such as a gastrointestinal cancer, or a pre-cancer state such as a colonic polyp or adenoma, and the targeting moiety is capable of binding a specific cellular marker of said tumor or pre-cancer state such as a cell surface receptor or a cell surface glycoprotein. In other particular such embodiments, the active agent adsorbed to the outer surface of the particles of the invention is an anti-EGFR antibody such as cetuximab, an EGF, or a lectin capable of binding galactosyl β-1,3-N-acetyl galactosamine.

In certain embodiments, the phospholipid-based particle of the invention comprises at least one NIR fluorescent probe and an active agent as defined above, both non-covalently bound to the outer surface of the particle, and one or more, e.g., two, three or four, additional active agents each independently non-covalently bound to the outer surface of the particle or embedded to the particle. In particular embodiments, the active agent non-covalently bound to the outer surface of the particle is a targeting moiety capable of selectively binding the particle to said target, and a further active agent, e.g., a therapeutic agent such as a chemotherapeutic agent, is either non-covalently bound to the outer surface of the particle or non-covalently embedded to the particle.

The particles of the present invention, whether comprising a water-insoluble polymer or a phospholipid, can be either nanoparticles or microparticles. The term “nanoparticle” as used herein refers to a particle as defined above having at least one dimension (such as width) which is a micron or smaller in size, preferably in the range of 20-300, more preferably 20-200, most preferably 20-150, nanometers, although other dimensions (such as length) may be longer than a micron. The nanoparticles may be of a uniform shape, e.g., spherical or elongated, or may have a variety of shapes. The term “microparticle” as used herein refers to a particle as defined above having at least one dimension (such as width) which is up to 200, preferably 150, more preferably 100, micrometers, although other dimensions (such as length) may be longer. The microparticles may be of a uniform shape, e.g., spherical or elongated, or have a variety of shapes, and may be aggregates of smaller particles.

In a further aspect, the present invention provides a powder comprising particles as defined above, i.e., either water-insoluble polymer-based or phospholipid-based particles. The powder of the invention can be prepared utilizing any available technology.

In certain embodiments, the powder of the invention comprises either freeze dried particles or spray dried particles. In particular embodiments, the powder of the invention further comprises one or more, i.e., one, two, three or more, cryoprotectants. Example of cryoprotectants that may be used in the process for the preparation of the powder include, without being limited to, sugars such as mannitol, sucrose and trihalose, polymers such as gum Arabic and polyvinyl pyrrolidone (PVP), proteins, and amino acids.

In still another aspect, the present invention provides a pharmaceutical composition comprising particles as defined above, i.e., either water-insoluble polymer-based or phospholipid-based particles, or a powder as defined above, and a pharmaceutically acceptable carrier.

The pharmaceutical compositions provided by the present invention may be prepared by conventional techniques, e.g., as described in Remington: The Science and Practice of Pharmacy, 19^{th} Ed., 1995. The compositions can be prepared, e.g., by uniformly and intimately bringing the active ingredient, i.e., the particles of the invention as defined above, into association with a liquid carrier, a finely divided solid carrier, or both, and then, if necessary, shaping the product into the desired formulation. The compositions may be in liquid, solid or semisolid form and may further include pharmaceutically acceptable fillers, carriers, diluents or adjuvants, and other inert ingredients and excipients.

The compositions can be formulated for any suitable route of administration, e.g., parenteral such as intravenous, intraarterial, intramuscular, intraperitoneal, intrathecal or subcutaneous, rectal, or inhalation administration, but they are preferably formulated for oral administration. The dosage will depend on the state of the patient, and will be determined as deemed appropriate by the practitioner.

The pharmaceutical composition of the invention may be in the form of a sterile injectable aqueous or oleagenous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution and isotonic sodium chloride solution.

Pharmaceutical compositions according to the invention, when formulated for oral administration may be in a form of tablets, troches, lozenges, aqueous, or oily suspensions, dispersible powders or granules, emulsions, hard or soft capsules, or syrups or elixirs. Pharmaceutical compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binders; and lubricating agents. The tablets are preferably coated utilizing known techniques to delay disintegration and absorption in the gastrointestinal tract and thereby provide an extended release of the active ingredient over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of bilayer tables, in which two or more distinct layers of granulation compressed together with the individual layers lying one on top of another, with each separate layer formulated to provide a different mode of release of the drug. Oral pharmaceutical composition of the invention may also be in the form of oil-in-water emulsion.

The pharmaceutical compositions of the invention may also be formulated as controlled-release matrix, e.g., as controlled-release matrix tablets in which the release of a soluble active ingredient is controlled by having the active ingredient diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or hydroxypropyl methyl cellulose, and among the different commercial grades of these ethers are those showing fairly high viscosity. In other configurations, the compositions comprise the active ingredient formulated for controlled release in microencapsulated dosage form, in which small droplets of the active ingredient are surrounded by a coating or a membrane to form particles in the range of a few micrometers to a few millimeters.

Another contemplated formulation is depot systems, based on biodegradable polymers, wherein as the polymer degrades, the active ingredient is slowly released. The most common class of biodegradable polymers is the hydrolytically labile polyesters prepared from lactic acid, glycolic acid, or combinations thereof.

The pharmaceutical composition of the invention may comprise one or more pharmaceutically acceptable excipients. For example, a tablet may comprise at least one filler, e.g., lactose, ethylcellulose, microcrystalline cellulose, silicified microcrystalline cellulose; at least one disintegrant, e.g., cross-linked polyvinylpyrrolidinone; at least one binder, e.g., polyvinylpyridone, hydroxypropylmethyl cellulose; at least one surfactant, e.g., sodium laurylsulfate; at least one glidant, e.g., colloidal silicon dioxide; and at least one lubricant, e.g., magnesium stearate.

In certain embodiments, the pharmaceutical composition of the invention, when formulated for oral administration, is in the form of a monolithic matrix, i.e., a structure including a three-dimensionally stable matrix material having a discrete size and shape; a tablet such as a bi-layered or multilayered tablet, matrix tablet, disintegrating tablet, dissolving tablet, or chewable tablet; or a capsule or sachet, e.g., filled with granules, grains, beads, or pellets. In other certain embodiments, the pharmaceutical composition of the invention, when formulated for oral administration, is in the form of a depot system, based on biodegradable polymers, wherein as the polymer degrades, the active ingredient is slowly released. The most common class of biodegradable polymers is the hydrolytically labile polyesters prepared from lactic acid, glycolic acid, or combinations thereof. Examples for biodegradable polymers prepared from these particular monomers include, without being limited to, poly(D,L-lactide) (PLA), polyglycolide (polyglycolic acid; PGA), and the copolymer poly(D,L-lactide-co-glycolide) (PLGA).

The term “controlled release”, “extended release”, or “sustained release”, as used herein interchangeably, refers to a mode of releasing an active ingredient from the formulation thereof such that it is absorbed by the body over a period of time. A controlled release formulation of an active ingredient may be accomplished, e.g., by embedding the active ingredient in a web of substance that the body is slow to dissolve, such that the active ingredient slowly and regularly leeches from the coating, or by swelling up the active ingredient to form a gel with a nearly impenetrable surface, wherein the active ingredient slowly exits the semipermeable layer.

In certain embodiments, the pharmaceutical composition of the invention, when formulated for oral administration, release of the active ingredient, i.e., the particles defined above, in a controlled release manner of zero, first, second or any other release profile (N^{th} order). The controlled release of the particles should preferably be slow and in certain embodiments the pharmaceutical composition is formulated so as to provide continuous sustained particle release, pulsatile particle release, multiphase particle release, or a combination thereof.

In certain embodiments, the pharmaceutical compositions of the invention, when formulated for oral administration, further comprise an enteric coating, i.e., a barrier that controls the location in the digestive system where it is absorbed, or in other words, prevents release of the active ingredient before it reaches the small intestine. Most enteric coatings work by presenting a surface that is stable at the highly acidic pH found in the stomach, but breaks down rapidly at a less acidic, i.e., relatively more basic, Materials used for enteric coatings include fatty acids, waxes, shellac, plastics, and plant fibers.

In particular embodiments, the enteric coating used in the pharmaceutical composition of the invention comprises a methacrylic acid copolymer such as Eudragit® L, Eudragit® S, Eudragit® RS, Eudragit® RL, Eudragit® FS 30P, and Eudragit® NE; or a cellulose derivative such as ethyl cellulose, cellulose acetophthalate, hydroxypropyl cellulose (HPC), hydroxypropylmethyl cellulose (HPMC), hydroxypropyl methylcellulose phthalate (HPMCP), and carboxymethylcellulose.

In one embodiment, the pharmaceutical composition of the invention, when formulated for oral administration, is used in detection and/or treatment of a gastrointestinal cancer or a pre-cancer state in the gastrointestinal tract.

Whereas conventional nanoparticles, when administered to various mucosal tissues, are likely to be trapped by mucus and rapidly eliminated via mucociliary clearance, the particles of the present invention more easily penetrate the mucus layer and have better chances to reach a target tissue, e.g., a colon epithelium. Internalization of targeting moiety-labeled particles has the added benefit of amplifying the fluorescent signal, since the fluorescent probe will accumulate in the tumor target cell. The enteric coating, if present, enables withstanding the acidic environment of stomach and digestive processes.

In one particular embodiment, the pharmaceutical composition is used for detection of a gastrointestinal cancer or a pre-cancer state in the gastrointestinal tract, wherein said at least one active agent non-covalently adsorbed to the outer surface of the particles of the invention is an anti-EGFR antibody such as cetuximab, an EGF, or a lectin capable of binding galactosyl β-1,3-N-acetyl galactosamine such as peanut agglutinin.

In another particular embodiment, the pharmaceutical composition is used for treatment of a gastrointestinal cancer or a pre-cancer state in the gastrointestinal tract, wherein one of said at least one active agent that is non-covalently adsorbed to the outer surface of the particles is an anti-EGFR antibody such as cetuximab, an EGF, or a lectin capable of binding galactosyl β-1,3-N-acetyl galactosamine such as peanut agglutinin; and another of said at least one active agent that is non-covalently bound or embedded to the particles is a chemotherapeutic agent.

In still a further aspect, the present invention thus relates to a method for detection of a cancer or a pre-cancer state in the gastrointestinal tract of an individual in need, said method comprising administering to said individual a pharmaceutical composition as defined above, formulated for oral administration, and detecting the presence of NIR emission from the walls of the gastrointestinal tract upon excitation at a proper wavelength.

In certain embodiments, the pharmaceutical composition administered according to this method comprises water-insoluble polymer-based or phospholipid-based particles as defined above, to the outer surface of which a NIR fluorescent probe and a targeting moiety capable of selectively recognizing a particular cellular marker of said cancer or pre-cancer state, thus binding the particles to the tissue of said cancer or pre-cancer state, are non-covalently bound. In particular embodiments, the NIR fluorescent probe bound to the outer surface of the particles is ICG, and the targeting moiety is an anti-EGFR antibody such as cetuximab, an EGF, or a lectin capable of binding galactosyl β-1,3-N-acetyl galactosamine such as peanut agglutinin.

Prior to administration of the pharmaceutical composition, the gastrointestinal tract of the individual treated may be purged using appropriate fluids, and following administration, suitable fluids may further be administered to rinse non-attached particles so as to ensure that NIR emission is detected essentially from regions having particles bounds through their targeting moieties only, i.e., regions containing cancer or a pre-cancer tissues.

The detection of NIR emission from the walls of the gastrointestinal tract according to the method of the invention may be carried out utilizing any suitable means, e.g., by means of colonoscopy or endoscopic pills, a tube with an optical fiber, a swallowed pill with a detector that transmits information to a receiver, or by a NIR detector that is placed outside the body.

In yet a further aspect, the present invention relates to a method for treatment of a cancer or a pre-cancer state in the gastrointestinal tract of an individual in need, said method comprising administering to said individual a therapeutically effective amount of a pharmaceutical composition as defined above, formulated for oral administration.

In certain embodiments, the pharmaceutical composition administered according to this method comprises water-insoluble polymer-based or phospholipid-based particles as defined above, to the outer surface of which a NIR fluorescent probe, a targeting moiety capable of selectively binding the particles to the tissue of said cancer or pre-cancer state, and a chemotherapeutic agent are non-covalently bound. In other certain embodiments, the pharmaceutical composition administered according to this method comprises water-insoluble polymer-based or phospholipid-based particles as defined above, to the outer surface of which a NIR fluorescent probe and a targeting moiety capable of selectively binding the particles to the tissue of said cancer or pre-cancer tissue are non-covalently hound, and a chemotherapeutic agent is non-covalently embedded to the particle.

In another aspect, the present invention relates to a method for the preparation of an aqueous dispersion of particles comprising a water-insoluble polymer, preferably a water-insoluble cationic polymer, to the outer surface of which at least one NIR fluorescent probe is non-covalently bound, said method comprising the steps of:
(i) dissolving a water-insoluble polymer in an organic solvent to obtain an organic solution of said polymer;
(ii) mixing said organic solution with an aqueous solvent in which a surfactant is optionally dissolved, in a ratio of 40:60 to 5:95, respectively, and removing said organic solvent to obtain an aqueous dispersion comprising particles of said polymer;
(iii) optionally mixing the aqueous dispersion of (ii) with an aqueous solution of a protein, peptide or polypeptide, to obtain an aqueous dispersion comprising particles of said polymer to the outer surface of which said protein, peptide or polypeptide is adsorbed;
(iv) mixing the aqueous dispersion of (ii) or (iii) with an aqueous solution of at least one NIR fluorescent probe thereby non-covalently binding said at least one fluorescent probe to the outer surface of said particles; and
(v) removing unbound molecules of said fluorescent probe from the dispersion obtained in (iv).

The organic solvent used in step (i) of this method so as to dissolve the water-insoluble polymer may be any suitable organic solvent such as acetone, ethanol, methanol, ethyl acetate, and acetonitrile, but it is preferably acetone.

In the study described herein, water-insoluble cationic polymer-based nanoparticles were prepared by the precipitation and solvent evaporation method, according to the following procedure: the cationic polymer Eudragit® RS was dissolved in acetone (10 wt %) and stirred by magnetic stirrer for at least 20 min; and the nonionic surfactant Pluoronic F-68 was dissolved in triple distilled water (5 wt %). The acetone solution and the aqueous solution, in a ratio of 20:80, respectively, were poured simultaneously into a vial and stirred immediately, and the acetone was then removed by evaporator. It is possible to control the particle's size by altering the preparation conditions, such as the surfactant concentration, the ratio between the organic and the aqueous solution, etc. Specifically, by using the above procedure, the particles obtained were spherical with an average size of 100 nm and zeta potential of +10 mV. When the Eudragit RS dispersion was diluted in an intestinal fluid (pH 6.8 with pancreatin), the particles were stable for at least two weeks.

The NIR dye adsorbed-Eudragit® RS nanoparticles were prepared by adding 100 of ICG stock solution (1.25×10⁻⁶ M in the final solution) to 6.3 ml of the Eudragit® RS nanoparticle dispersion, followed by mild shaking for one hour. The obtained dispersion was then filtered by centrifugation in a 300 kDa filtration tube for 10 min at 40 rpm, so as to remove the free ICG. The filtrate did not contain ICG, while the retained nanoparticles had green color, indicating that the ICG was attached to the nanoparticles by non-covalent interactions. The emission of the retained nanoparticles was measured by Cary Eclipse fluorimeter (exitation at 780 nm; emission at 800 nm).

In a further aspect, the present invention relates to a method for the preparation of an aqueous dispersion of particles comprising a phospholipid, to the outer surface of which at least one NIR fluorescent probe and at least one active agent are non-covalently bound, said method comprising the steps of:
(i) providing an aqueous dispersion of particles comprising a phospholipid;
(ii) mixing the aqueous dispersion of (i) with an aqueous solution of at least one NIR fluorescent probe thereby non-covalently binding said at least one fluorescent probe to the outer surface of said particles;
(iii) removing unbound molecules of said fluorescent probe from the dispersion obtained in (ii);
(iv) mixing the aqueous dispersion of (iii) with an aqueous solution of said at least one active agent thereby non-covalently binding said at least one active agent to the outer surface of said particles; and
(v) removing unbound molecules of said at least one active agent from the dispersion obtained in (iv).

The steps of removing unbound molecules of the fluorescent probe or unbound molecules of the active agent may be carried out by any suitable technique, such as by filtration or centrifugation. The size of the particles prepared according to this method can be controlled by altering one or more of the preparation conditions.

In the study described herein, phospholipid-based nanoparticles were prepared according to the following procedure: lecithin 5% w/w was dispersed in deionized water and stirred by a magnetic stirrer at 40° C. for 40 min, followed by sonication for 10 min, and the resulting dispersion was diluted by phosphate buffer to a final concentration of 1%. All preparations were performed under nitrogen. This procedure is different from the conventional method in which the phospholipid is solubilized in an organic solvent. The average size of the phospholipid particles obtained was ˜30 nm and their zeta potential was of −50 mV.

The NIR dye adsorbed-phospholipid nanoparticles were prepared by adding 50 μl of ICG stock solution (3.2×10⁻³ M in the final solution) to 5 ml of the phospholipid nanoparticle dispersion followed by mild shaking for one hour. The obtained dispersion was then filtered by centrifugation in a 300 kDa filtration tube for 10 min at 40 rpm, so as to remove the free ICG. The filtrate did not contain ICG, while the retained nanoparticles had green color, indicating that the ICG was attached to the nanoparticles by non-covalent interactions. The emission of the retained nanoparticles was measured by Cary Eclipse fluorimeter (exitation at 720 nm; emission at peak). It should be noted that the ICG-adsorbed phospholipid particles were more fluorescent relatively to free ICG in range of 6.2×10⁻⁷-9.6×10⁻⁵ M. It seems that the phospholipid particles did not only bind ICG but also protect it from possible thermal or light degradation and aggregation in room temperature. These findings are even more impressive in view of the fact that according to previous reports, liposomes trigger cyanine dyes aggregation, which reduces fluorescence.

Active molecule-labeled ICG-adsorbed phospholipid nanoparticles were prepared by binding a targeting molecule such commercially available IgG-FITC, EGF, cetuximab and PNA, which can be detected by fluorescence or ELISA measurements. In particular, a targeting agent dissolved in appropriate buffer was added to ICG-adsorbed phospholipid nanoparticle dispersion and the dispersion obtained was shaken for 24 hours, and was then filtered by filtration tube of 300 kDa for 10 min at 40 rpm so as to remove the free targeting agent. IgG-FITC, cetuximab and EGF passive attachment was analyzed by ELISA assay, and the passive attachment of PNA-FITC was confirmed by fluorescence measurements.

In summary, the approach described in the study described herein refers to preparation of multimodal particles, herein also identified “nanoprobes” or “microprobes”, capable of specifically binding to cancer cells and detected by a NIR fluorescence probe, preferably ICG that is already in clinical use. Both the fluorescence probe and the targeting moiety are non-covalently bound, i.e., adsorbed, to the particles.

The liposomal nanoprobes exemplified herein have an average size of 30 nm and zeta potential of −30 mV. It seems that ICG was passively adsorbed to the liposomes through electrostatic interactions, because the zeta potential decreased in absolute value to about −15 mV. An electrostatic mechanism of adsorption was also reported for other cyanine dyes (DeRossi et al., 1996). Liposomal ICG was more fluorescent than the free ICG in solution, probably as a result of the higher monomeric fraction of ICG in the liposomal form. A similar phenomenon was reported for another cyanine dye, 3,3-disulfobutyl-5,5-diphenyl-9-ethyl-oxacarbocyanine, in the presence of phosphatidylserine (Sidorowicz et al., 1997).

As shown in the binding isotherm and the decrease in zeta potential, increasing the concentration of ICG leads to a greater concentration of ICG molecules bound to the liposome. This is expected to enable closer packing of the ICG molecules, and thus a greater probability for aggregation of the dye molecules, resulting in a red shift and quenching. The shift in fluorescence might be explained by the less polar environment (DeRossi et al., 1996; Sidorowicz et al., 1997) and formation of ICG aggregates that emit at a higher wavelength (Saxena et al., 2003; Sidorovicz et al., 2005).

As further found, the liposomal ICG had an approximately threefold higher quantum yield than that of a simple solution of ICG, indicating a significant improvement in detection capabilities. Similarly, an increase in quantum yield ratio was also observed in a micellar system (Kirchherr et al., 2009). As reported for other cyanine dyes (DeRossi et al., 1996), the greater quantum yield can be explained by the higher order of the molecules in the liposomes and by the more rigid structure, which prevents free molecular motion and preferable adsorption in monomeric form (Philip et al., 1996). The high stability of the liposomal nanoprobes in the colon opens a way for accelerated clinical use.

The IgG molecule was used as a model targeting agent that can be passively adsorbed to the liposomal surface, and the ELISA results indicated passive absorbance of IgG molecules. In order to support the conclusions from the ELISA measurements, and to evaluate whether ICG-adsorbed liposome binding antibodies retain their NIR fluorescence, the fluorescence of both ICG and FITC was measured. As found, the fluorescence of both ICG and FITC was detected in the fraction of the solution that was retained after filtration, indicating that both ICG and IgG are bound to the liposomes. The physical proximity of ICG and IgG as obtained in liposomes resulted in conservation of ICG fluorescence, in contrast to the disappearance of its fluorescence while it is covalently bound to IgG (Ogawa et al., 2009). The retention of ICG fluorescence in the presence of IgG and increasing its quantum yield in liposomes indicates the advantage of the binding through simple adsorption methodology.

Cetuximab-labeled ICG-adsorbed liposomes kept their EGFR-specific recognition ability as was shown by 10-fold higher fluorescent signal relative to control, untargeted liposomes. Interestingly, that analysis revealed that a larger fraction of cetuximab-labeled liposomes was incorporated within the intracellular compartment relative to IgG-labeled liposomes, which were mainly membrane associated. This difference in the membrane-to-cytosol ratio indicates an EGFR-mediated specific process associated with cetuximab-labeled liposomes. Internalization of cetuximab-labeled liposomes has the added benefit of amplifying the fluorescent signal, because the fluorescent probe will accumulate in the tumor target cell. Therefore, adsorption of ICG and cetuximab to liposomes provides a practical approach for generating a tool for imaging of carcinomas overexpressing EGFR. Another approach for imaging is based on covalent attachment of ICG molecules directly to an antibody (Ogawa et al., 2009; Withrow et al., 2007). However, the covalent attachment approach has several major drawbacks: (i) It was found that the ICG significantly loses its fluorescence activity as a result of the chemical binding (Ogawa et al., 2009); (ii) The number of ICG molecules per antibody was limited to only five (Ogawa et al., 2009) or one (Withrow et al., 2007), whereas liposomes may bear potentially larger ICG quantities; (iii) The ICG-antibody conjugate is obtained by covalent attachment, and therefore the resulting molecule is a new moiety that would require an additional approval process before it can be utilized in clinics. The approach presented herein, according to which both ICG and cetuximab are physically adsorbed onto the liposome, overcomes these drawbacks and provides a new and simple paradigm for EGFR-overexpressing tumor imaging.

Nonspecific binding is a critical issue for noninvasive tumor imaging. Therefore, NIR imaging of living cells was used for evaluation of the specificity of the nanoprobes. The signal ratio of A431/IEC6 was 3.5 for cetuximab-labeled liposomes, whereas for unlabeled liposomes it was 40% lower, thus indicating more pronounced nonspecific binding. Cetuximab-labeled liposomes enhanced the signal-to-background ratio by decreasing the level of the background labeling of the cells as opposed to nonspecific IgG-labeled liposomes. The variation in signal strength between the binding of cetuximab and that of IgG-labeled liposomes, measured with carcinoma cultures in vitro, has the potential to predict the usefulness of this approach in future in vivo experiments. Indeed, liposomes conjugated with EGFR ligand peptides (Song et al., 2009) or cetuximab (Mamot et al., 2005) were able to bind to EGFR in in vivo experiments, thus further supporting the usefulness of ICG-loaded cetuximab-labeled liposomes for in vivo targeting. Liposomes hold tremendous potential as diagnostic imaging pharmaceutical tools and promise for the development of multimodality agents with both imaging and therapeutic capabilities. The creation of biocompatible liposomes will provide an advanced enabling technology for diagnosis and therapy of carcinomas based on EGFR and its oncogenes.

The cationic polymer-based nanoprobes exemplified herein have an average size in the range of 50-300 nm and zeta potential above +5 mV. ICG is passively adsorbed to the cationic nanoparticles, probably but not necessarily, by electrostatic interactions. This assumption is based on experiments in which ICG aqueous solution was mixed with ion exchanger (anions and cations), following which the uncolored beads became green. Interestingly, the non-covalent binding of the ICG to the nanoparticles improved the fluorescence properties and intensity, as well as the stability of the ICG.

As shown in the binding isotherm, increasing the concentration of ICG leads to a greater concentration of ICG molecules bound to the cationic polymer-based particle. It should be noted that at high ICG concentrations, aggregation of the nanoparticles occurred. The maximal concentration of ICG at which there was no aggregation was 10⁻⁵ M. At this concentration the amount of adsorbed ICG molecules was approximately 10⁴ ICG molecules per nanoparticle (Yuan et al., 2004). These improved features are essential for achieving strong and clear signal from the detected tissue.

As further found, cationic polymer-based particles loaded ICG had an approximately fivefold higher quantum yield than that of a simple solution of ICG, indicating a significant improvement in detection capabilities.

The IgG molecule was used as a model targeting agent that can be passively adsorbed to the cationic polymer-based particles surface, and the ELISA results indicated passive absorbance of IgG molecules. In order to support the conclusions from the ELISA measurements, and to evaluate whether ICG-adsorbed cationic polymer based particles binding antibodies retain their NIR fluorescence, the fluorescence of both ICG and FITC was measured. As found, the fluorescence of both ICG and FITC was detected in the fraction of the solution that was retained after filtration, indicating that both ICG and IgG were bound to the cationic polymer-based particles. The physical proximity of ICG and IgG as obtained in cationic polymer-based particles resulted in conservation of ICG fluorescence, in contrast to the disappearance of its fluorescence while it is covalently hound to IgG (Ogawa et al., 2009). The retention of ICG fluorescence in the presence of IgG and increasing its quantum yield in cationic polymer-based particles indicate the advantage of the binding through simple adsorption methodology.

Negatively charged sodium caseinate (a blocking molecule) was non-covalently absorbed onto the ICG-loaded cationic polymer-based particles. Those particles have an average size of 200 nm and zeta potential of −29 mV. Stability evaluation conducted in human colon fluids indicated that the ICG-loaded particles to which the caseinate was adsorbed are stable even after 7 hours in 37° C. The high stability of these particles in the colon opens a way for accelerated clinical use.

In order to obtain specific recognition of the negatively charged cationic polymer-based particles to colorectal tumors, different types of targeting molecules, in particular, cetuximab that is approved for medical use, as well as peanut agglutinin and anti-CEA, were physically adsorbed to the surface of those particles. The adsorption of the different targeting molecules was characterized by adsorption isotherms, prepared by ultrafiltration, centrifugation and ELISA.

The binding ability of the negatively charged cationic polymer-based nanoprobes to colorectal tumors was further evaluated in vivo and ex vivo, using various biological models. In vivo binding tests in mice (LS174T tumor bearing mice) and rats (DMH-treated rat colon) exemplified herein, as well as experiments conducted using a chicken embryo model with LS174T and HT29 cells (data not shown), showed that these particles, either with or without a targeting molecule, were capable of binding in vivo with high specificity (high signal to background ratio).

Cationic polymer-based particles hold tremendous potential as diagnostic imaging pharmaceutical tools and promise for the development of multimodality agents with both imaging and therapeutic capabilities.

The invention will now be illustrated by the following non-limiting Examples.

### EXAMPLES

### Abbreviations

AFM, atomic force microscopy; CEA, carcinoembryonic antigen; DLS, dynamic light scattering; DMEM, Dulbecco's Modified Eagle Medium; DMH, dimethylhydrazine; EGF, epidermal growth factor; EGFR, epidermal growth factor receptor; ELISA, enzyme-linked immunosorbent assay; FCS, fetal calf serum; FITC, fluorescein isothiocyanate; HRP, horseradish peroxidase; FIR-SEM, high-resolution scanning electron microscopy; ICG, Indocyanine green; NIR, near infrared; PBS, phosphate buffered saline; PNA, peanut agglutinin; SEM, scanning electron microscopy; TIM, triple distilled water; TEM, transmission electron microscopy; TMB, 3,3′,5,5′-tetramethylbenzidine.

### Material and Methods

### Materials

The following materials were used with no further purification: ICG (IR-125, Laser Grade) (Holland Moran, Yehud, Israel); Phospholipon 50 by Lipoid (Ludwigshafen, Germany); Eudragit RS-100 granules (Degussa-Rohm, Piscataway, N.J., USA); acetone technical (Bio-Lab, London, UK); Pluronic F-68, casein sodium salt from bovine milk, sodium caseinate, fluorescein isothiocyanate (FITC) and FITC-human immunoglobulin G (IgG) (Sigma-Aldrich, Rehovot, Israel); PNA-FITC (PNA-FITC) (Vector Labs); fluorescein dilaurate (Fluka, Exeter, UK); cetuximab was provided by the Pharmacy Unit of the Hadassah-University Medical Center; human IgG enzyme-linked immunosorbent assay (ELISA) kit E-80G (ENCO, Petach Tikvah, Israel); Human recombinant epidermal growth factor (EGF) (Peprotech-Asia, Rehovot, Israel); antibodies against phospho- or pan-Erk1/2 (extracellular-signal-regulated kinase) (Cell Signaling Technology Inc., Beverly, Mass., USA); HRP-conjugated goat anti-rabbit secondary antibody (Jackson ImmunoResearch, West Grove, Pa., USA); and anti-CEA (T84.66) (Alomon Labs).

### General

All the experiments described in the Examples below were carried out according to the procedures described in Materials and Methods unless otherwise stated.

### Liposome (Phospholipid Nanoparticles) Preparation

Phospholipon 50 5% (wt/wt) was dispersed in deionized water and stirred using a magnetic stirrer at 40° C. for 40 min, followed by sonication for 10 min (Hielscher UP200S; Teltow, Germany; 60 Hz, cycle 0.5). The resulting dispersion was diluted in phosphate buffer to a final concentration of 1%. All preparations were performed under N₂. All experiments were carried out with 5 mM phosphate buffer (pH 7) unless otherwise stated.

### Polycationic Nanoparticles Preparation

Polycationic nanoparticles were prepared by precipitation and solvent evaporation according to the following procedure: Eudragit RS 100, a copolymer of ethyl acrylate, methyl methacrylate and a low content of methacrylic acid ester with quaternary ammonium groups (MW 150,000, see Scheme 1), was dissolved in acetone (10 wt %) and stirred by magnetic stirrer for at least 20 min. Pluronic F-68, a nonionic diblock copolymer surfactant serving as a dispersing agent, was dissolved in TDW (5 wt %). The Pluronic F-68 was chosen due to its low toxicity and its known capability to stabilize dispersions (Bogdanova and Dolzhikova, 2008). The acetone and aqueous solutions were poured simultaneously into a vial while stirring with a magnetic stirrer. The acetone/aqueous solutions ratio was 20:80, respectively. The acetone was removed by evaporation at room temperature.

### Binding of ICG to the Particles

ICG (see Scheme 1) stock solution of 3.2×10⁻³ M was prepared by dissolving 60 mg ICG in 25 ml of deionized water.

Binding to Liposomes.

The binding of ICG to the liposomes was performed by addition of various quantities of ICG stock solution to 700 μl of 1% (wt/wt) liposomal dispersion; a final volume of 3.2 ml was achieved with buffer. The dispersion was incubated under mild agitation at 5° C. for 24 hrs.

Binding to Polycationic Particles.

The binding of ICG to the polycationic nanoparticles was performed by addition 100 μl of ICG solution at various concentrations (prepared from the stock solution) to 6.3 ml of the nanoparticle dispersion, followed by mild shaking for 1 hr.

The quantity of ICG adsorbed onto the nanoparticles was calculated after determination of the free ICG present in the aqueous phase of the dispersion. The ICG dispersion was filtered through a 300-kDa filtration tube (VS0241 VIVA SPIN; Beit Haemek, Israel) for 10 min at 40 rpm using a Centrifuge CN-2200 (MRC, Holon, Israel) to collect the aqueous phase of the dispersion (filtrate).

In the case of the liposomes, the free ICG was determined by absorbance spectra, using a Cary 100 Bio spectrophotometer (Varian, Palo Alto, Calif.) in a 1-cm polystyrene cell at a scan rate of 600 nm/min. In the case of the polycationic nanoparticles, the ICG concentration in the filtrate was determined by measurement at 780 nm. The binding isotherm was obtained by performing a series of such experiments, followed by plotting the quantity of adsorbed ICG as a function of the concentration of the free ICG. Based on the fluorescence results, the obtained particles were used for adsorption of targeting molecules in the following experiments.

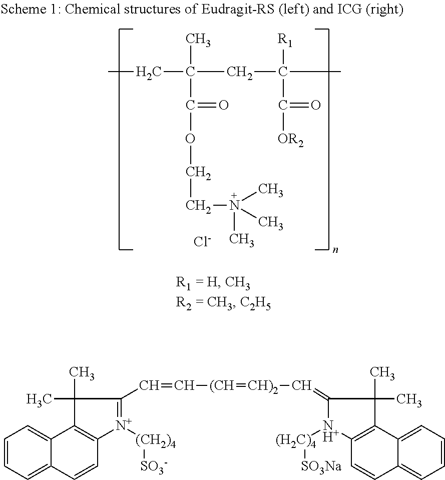

### Binding of IgG-FITC to the ICG-Adsorbed Particles

ICG-Adsorbed Liposomes.

The ICG was incubated with 1% liposomal dispersion under mild agitation at 5° C. for 24 hrs; the final ICG concentration was 3.2×10⁻² mM. 100 μl of FITC-IgG (1 mg/ml) were added to 1.5 ml of the liposomal dispersion, followed by mild agitation at 5° C. for 24 hrs. The dispersion was then filtered (filtration tube of 300 kDa for 10 min at 40 rpm) with washing 3 times with 1 ml of buffer to remove the free IgG.

ICG-Adsorbed Polycationic Nanoparticles.

50 μl of FITC-IgG (20 mg/ml, Sigma) were added to 5 ml of the ICG-adsorbed polycationic nanoparticle dispersion, followed by mild shaking for 2 hrs. Next, 2 ml of the dispersion was filtered (filtration tube of 300 kDa for 15 min at 60 rpm with water washing three times with 1 ml of water each time) to remove the free IgG.

### ELISA Experiments

ELISA experiments were performed with the human IgG ELISA Kit (E-80G; ENCO) according to the manufacturer's recommendations. In particular, to commercial ready-to-use 96-well plates containing capture antibody, 100 μl/well of the samples (IgG-FITC-labeled ICG-adsorbed nanoparticles) and controls (Human IgG) were added and incubated for 1 hr. At the next step, consecutive washings were performed and a primary anti-IgG antibody conjugated with HRP was added (100 μl/well) and incubated for 20 min. Following an additional washing step, the enzyme bound to the immunosorbent was assayed by the addition of 100 μl/well of TMB substrate. After 10 min the reaction was stopped by addition of sulfuric acid (0.3 M). After 10 min the absorbance was detected at 450 nm using an ELISA plate reader (Tecan, Switzerland).

### Absorbance and Fluorescence Measurements

Absorbance spectra of the samples were taken using a Cary 100 Biospectrophotometer, in a 1 cm polystyrene cell, and a scan rate of 600 nm/min.

Fluorescence spectra of the samples were taken using a Cary Eclipse fluorimeter (Varian), a 1-cm quartz cell, and a scan rate of 600 nm/min. In the case of the liposomes, measurements were performed at λₑₓ 720 nm; slits for both emission and excitation were 10 nm. For relative quantum yield λₑₓ and λ_{ubs} were 720 nm; slit for excitation was 5 nm and for emission 20 nm. In the case of the polycationic nanoparticles, excitation and emission slits were both fixed at 5 nm, and λₑₓ was 780 nm for ICG and 800 nm for ICG-loaded nanoparticles. In the FITC experiments, the excitation and emission slits were fixed on 2.5 nm, and λₑₓ was 490 nm.

### Size Measurement/Light Scattering

Size measurements were performed with a Zetasizer Nano-S (Malvern Instruments, Worcestershire, UK). The aqueous dispersions were measured without dilution. Liposome aggregates were visualized using an MEE-643 light microscope (Ueda, Germany). In the case of the polycationic nanoparticles, the calculation of size distribution from light scattering measurements was based on the assumption that the particles are spherical, and the refractive index of the particles is 1.49 (Seferis, 1999).

### Zeta Potential

Zeta potential measurements were performed with a Zetasizer Nano-S (Malvern Instruments). The liposome samples were measured after dilution (200 μl in 4 ml of buffer), and the polycationic nanoparticle samples were measured diluted in 10 mM NaCl solution.

### Cryo-TEM

The liposome nanoparticles were imaged using cryo-TEM. Samples were prepared in the controlled-environment vitrification system (Bellare et al., 1988) at 25° C. and at water saturation to avoid evaporation. Specimens were studied in a Philips CM120 TEM (Philips, Eindhoven, The Netherlands) operating at 120 kV with an Oxford CT3500 (Oxford Instruments, Abingdon, Oxfordshire, UK) cryoholder, maintained below −172° C. Digital images were recorded on a cooled Gatan MultiScan 791 charge-coupled device camera (Gatan, Abingdon, Oxfordshire, UK) using the Digital Micrograph 3.1 software (Gatan). Imaging was done in the low-dose mode to minimize beam exposure and electronbeam radiation damage (Damino et al., 2001).

### HR-SEM

The polycationic nanoparticles were imaged using a HR-SEM (Sirion HR-SEM, FEI Company, Hillsboro, Oreg., USA).

### Stability in Colon Fluid

The stability of the ICG-adsorbed liposomes in human colon fluid was evaluated as follows. 9 ml of colon content from two post-op patients were centrifuged, filtered (5 μm and 0.45 μm), and added to 500 μl of liposomal dispersion (1% w/w, ICG 3.2×10⁻² mM). The dispersions were incubated under mild agitation at 37° C. for 7 hrs. The dispersions were filtered by ultrafiltration test tubes (300 kDa) (VS0241 VIVA SPIN, Beit Haemek, Israel), and the fluorescence of the supernatant and the filtrate were evaluated by the Odyssey NIR scanner system (Li-Cor, Lincoln, Nebr., USA).

### Stability Test in Intestinal Fluid

A total of 10 ml of ICG-adsorbed polycationic nanoparticles (10⁻⁴ M, final concentration) and 37.5 μl of IgG-FITC (0.075 mg/ml) were added to 200 ml of simulated intestinal fluid (USP26) at 37° C., followed by mild shaking. Next, 1.5 ml of the dispersion was diluted 1:1 v/v with deionized water and filtered to remove the free ICG and the free IgG (filtration tube of 300 kDa for 10 min at 15 rpm). A total of 1 ml of the filtrate was diluted 1:3 v/v with deionized water. The ICG absorbance and fluorescence of IgG-FITC was followed over 8 hrs. The excitation and emission slits were fixed both at 20 nm and λₑₓ was 480 nm.

### Cell Culture

A431 colon carcinoma cells overexpressing EGFR and IEC-6 colon cells (normal enterocytes expressing physiological EGFR levels) were cultured in T75 tissue culture flasks (Nunc, Denmark). The growth medium consisted of DMEM supplemented with 4.5 mg/ml glucose, 10% FCS, 2 mM 1-glutamine, penicillin 10,000 U/ml, and streptomycin 100 μg/ml (Beit Haemek, Israel). Confluent cultures were split at a 1:10 ratio after trypsinization with 0.25% trypsin solution (Beit Haemek, Israel). All cell cultures were maintained at 37° C. in a humidified incubator in a mixture of 5% CO₂/95% air. The medium was changed every second day. Visual evaluation of the cells was performed using TS-100 inverted phase contrast light microscope (Nicon® Eclipse, Japan).

### Confocal Microscopy

For visualization of liposomes binding to EGFR, cetuximab or IgG solutions (0.01 mg/ml in buffer) were added 1:1 to 1% wt/wt liposomal dispersion, and the unlabeled liposomes were dispersed similarly in buffer. The dispersion was incubated under mild agitation at 5° C. for 24 hours. 50 μl of fluorescein dilaurate (1 mg/ml) in pure ethanol were added to 0.5% wt/wt liposomal dispersion, mixed for 3 hrs at room temperature 22-25° C., and then filtered through a 300-kDa filtration tube and washed three times with buffer. For cell labeling, A431 cells were placed on cover slides and left overnight to adhere. The following day the adherent cells were incubated with EGF-FITC or liposome solutions for 30 min followed by three washes with PBS. Thereafter, the cells were fixed with 4% paraformaldehyde (Sigma-Aldrich) and washed three times with PBS. In the negative control experiments, the EGF-FITC or liposome solution incubation step was omitted while the other steps remained the same. The cells were examined in a FluoView FV300 confocal laser scanning microscope (Olympus, Tokyo, Japan).

### In Vitro NIR Imaging Using Odyssey Scanner

Liposomal Dispersion.

The ICG with 1% liposomal dispersion was incubated under mild agitation at 5° C. for 24 hrs; the final concentration of ICG was 3.2×10⁻² mM. Cetuximab solution (0.01 mg/ml) was added 1:1 to 1% wt/wt liposomal dispersion; in parallel, the unlabeled liposomes and IgG-labeled liposomes were similarly diluted in buffer. The dispersion was incubated under mild agitation at 5° C. for 24 hrs. To start the binding, cultures were incubated with liposome solutions for 30 min. At the end of incubation, the cultures were washed 3 times with PBS and imaging was performed (the focal cell model is described hereinbelow). The tissue culture plates were placed inside an Odyssey NIR laser scanner (Li-Cor; Lincoln, Nebr.). The plates were scanned with fixed intensity at excitation 785 nm and emission 800 nm. The intensity of scanning was calibrated with free liposome solution (data not shown) and was kept constant during all experiments to avoid overflow values. The obtained images were quantified using the Li-Cor imaging program provided with the scanner. The results presented as the mean±SD of at least three independent experiments (n=9-15) were evaluated using the InStat3 statistics program (GraphPad, La Jolla, Calif.). Statistically significant differences between experimental groups were determined by analysis of variance with Bonferroni post hoc test, with P<0.05 considered significant.

Polycationic Nanoparticle Dispersion.

The possible applicability of the NIR polycationic nanoparticles for remote imaging, which is based on NIR fluorescence, was evaluated by imaging the ICG through a layer mimicking human tissue. Therefore, pastrami slices (up to a thickness of 1.6 cm) were placed under test tubes containing ICG aqueous solution (5×10⁻⁶ M) or a dispersion of ICG-adsorbed naoparticles (ICG concentration 5×10⁻⁶ M). The fluorescence through the pastrami was measured by Odyssey NIR laser scanner (Li-Cor, NE, USA; excitation: 785 nm; and emission: 800 nm).

### Focal Cell Model of Colon Cancer

The in vitro NIR imaging was performed in a focal model of colon cancer. To generate the in vitro model, 15×10³ A431 cells were plated at the center of a 12-well tissue culture plate inside a 4-mm inner diameter cloning ring. Upon plating, the A431 cells were left to adhere for 2 hrs in an incubator. Thereafter, 15×10³ IFC-6 cells were plated in the surrounding free area and left to adhere for 2 hrs. At the end of cell adherence phase, the cloning ring was removed and the cells were washed three times with culture medium. Two days later, the cultures were subjected to binding and imaging experiments. Before the binding experiments the cultures were washed three times with DMEM without serum (binding medium). The cells were incubated with the binding medium for 2 hrs before initiation of the experiments.

### Western Blotting

Western blotting was performed as described by Lecht et al. (2010). Briefly, total cellular protein was extracted following lysis of the A431 cells and quantified using the Bradford reagent assay (Bio-Rad, Hercules, Calif., USA). For Western blotting, equal quantities of protein (40 μg) were loaded on 10% polyacrylamide gels, separated by SDS-PAGE (100 V for 1.5 hrs), and transferred on ice to nitrocellulose membranes (90 V for 1.5 hrs) (Whatman, Germany). Immunodetection was performed using primary antibodies against phospho- or pan-Erk1/2 (both antibodies: 1:1000; Cell Signaling Technology Inc., Beverly, Mass., USA). The detection was performed using an HRP-conjugated goat anti-rabbit secondary antibody (1:10,000; Jackson ImmunoResearch, West Grove, Pa., USA).

Synthesis and Purification of EGF Labeled with FITC

The covalent attachment of human recombinant EGF with FITC was performed as previously described (Haigler et al., 1978) with several modifications. In particular, to initiate the reaction, 250 μl of 0.33 mg/ml FITC solution in 0.2M NaHCO₃ was slowly added during 1 hr to 100 μl of 5 mg/ml EGF solution in 0.2M NaHCO₃, and was gently stirred for 10 hrs in a conical glass tube with low protein absorption properties. Thereafter, another 250 μl of FITC solution was slowly added to the reaction tube and left for an additional 12 hrs of gentle stirring. In order to separate the FITC-labeled EGF from excess FITC and unlabeled EGF, two steps of purifications were performed. At the first step, the free FITC was separated using desalting procedure. The solution was loaded on a Hiprep 26/10 column and separated using an FPLC AKTA P900 instrument (GE Healthcare-Life Sciences, UK). The mobile phase was composed of double distilled water; the flow rate was 10 ml/min and the pressure 0.15 mPa. The protein-containing fraction was collected based on detection at 280 nm and 495 nm absorption using an automatic sample collector. The collected fraction was dried in a lyophilizer for 24 hrs. At the second step, the labeled and unlabeled EGF were separated based on ion exchange principle using Hitrap DEAE FF column (GE Healthcare-Life Sciences, UK). The lyophilized product was dissolved in 1 ml of Tris-base 20 mM (pH 8). The mobile phase was composed of Tris-base 20 mM (pH 8) and the FPLC instrument was set up at a flow rate of 1 ml/min and pressure of 0.3 mPa. After loading the sample on the column the elution was performed at 0.75M NaCl gradient 1-100% at a rate of 5%/min. The detection of the proteins was performed at 280 nm and 495 nm, and the fractions with absorption at both wavelengths, indicating presence of EGF attached to FITC, were collected. The collected fractions were further purified using dialysis sacks with 3000 Da cut-off (Thomas scientific, USA) against distilled water for 14 hrs at 4° C. with constant stirring. Thereafter the content of the dialysis sack was lyophilized and kept in the dark at 4° C.

### Example 1

### Preparation of ICG-Adsorbed Liposomes

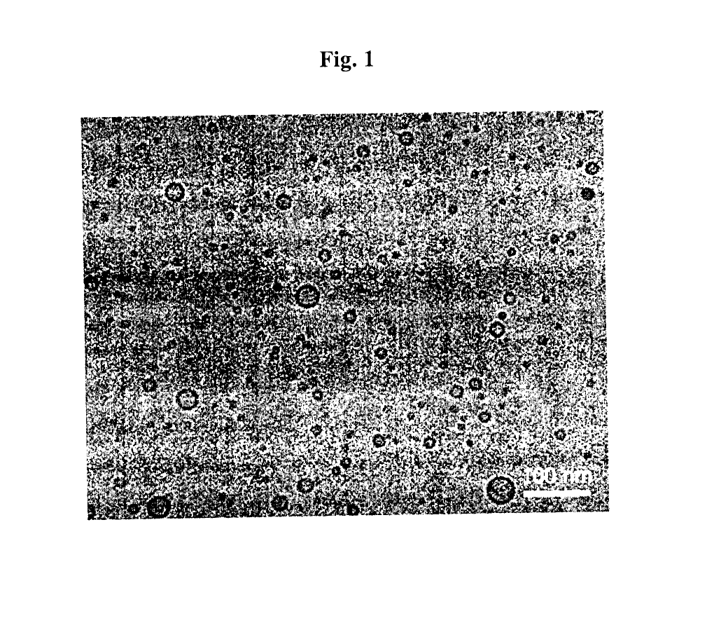

Liposomes composed of Phospholipon 50 (Lipoid), permitted for oral delivery, were prepared by high-energy sonication, which typically leads to formation of small unilamellar liposomes. DLS measurements showed that the mean size (by volume) of the liposomes is 29.8±0.6 nm with a polydispersity index of 0.235-0.250, and zeta potential of −30.5±1 mV (at pH 7). Cryo-TEM imaging, shown in FIG. 1, confirmed that the liposomes are unilamellar in the size range of 20-40 nm, in agreement with DLS results.

The ICG binding experiments were performed by incubation of the liposomes with solutions of ICG at various concentrations. The adsorbed quantity of ICG was calculated by measuring the optical density of the solution obtained after filtration of the samples by a 300-kDa filter, which is expected to remove all the liposomes. The retained particles had a green color, giving a visual indication that the ICG is bound to the liposomes.

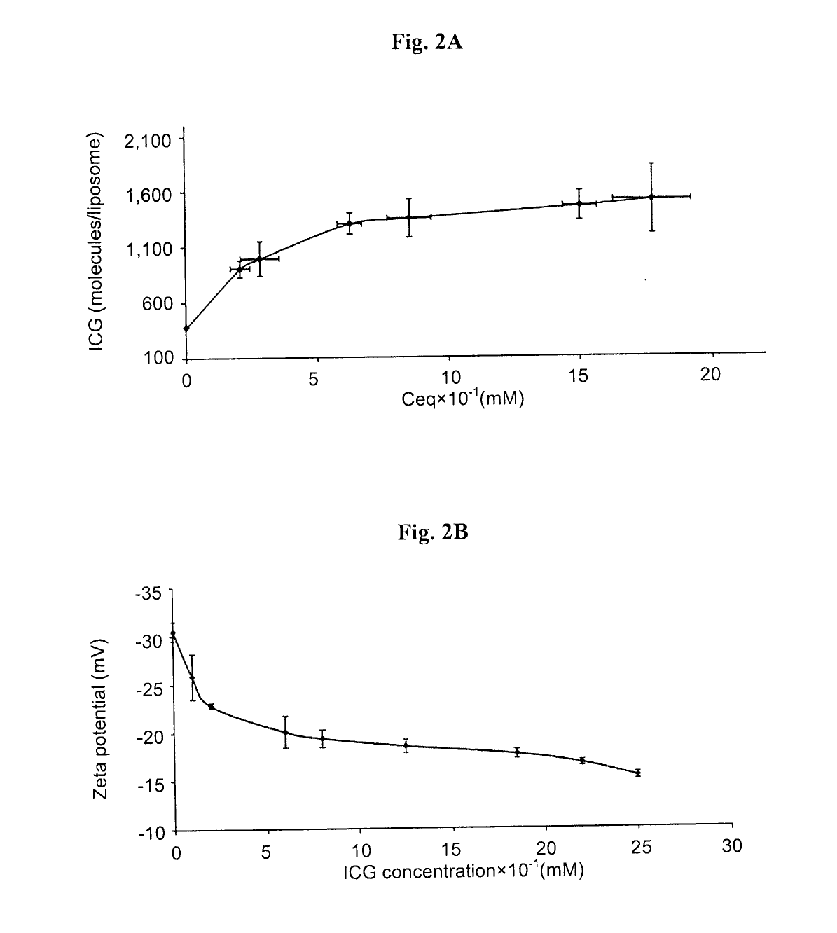

As shown in the adsorption isotherm in FIG. 2A, the quantity of adsorbed ICG increased with the increase in ICG concentration in solution. The liposome number was calculated according to Jones (2005). A hilayer thickness was taken as 4 nm, and a cross-sectional area of phospholipid as 0.71 nm² (Torre et al., 2007). It should be noted that high ICG concentrations caused aggregation of liposomes, and the maximal concentration of ICG that did not cause aggregation was 46 molecules per liposome. For the greatest ICG concentration in the adsorption isotherm (1519 molecules per liposome), the aggregate size, as seen by optical microscope, is 1.4-4.2 μm (data not shown). In addition to the size increase, it was found that binding ICG caused a decrease in the absolute value of zeta potential from mV to −15.35±0.35 mV, as shown in FIG. 2B.

### Example 2

### Fluorescence of the ICG-Adsorbed Liposomes

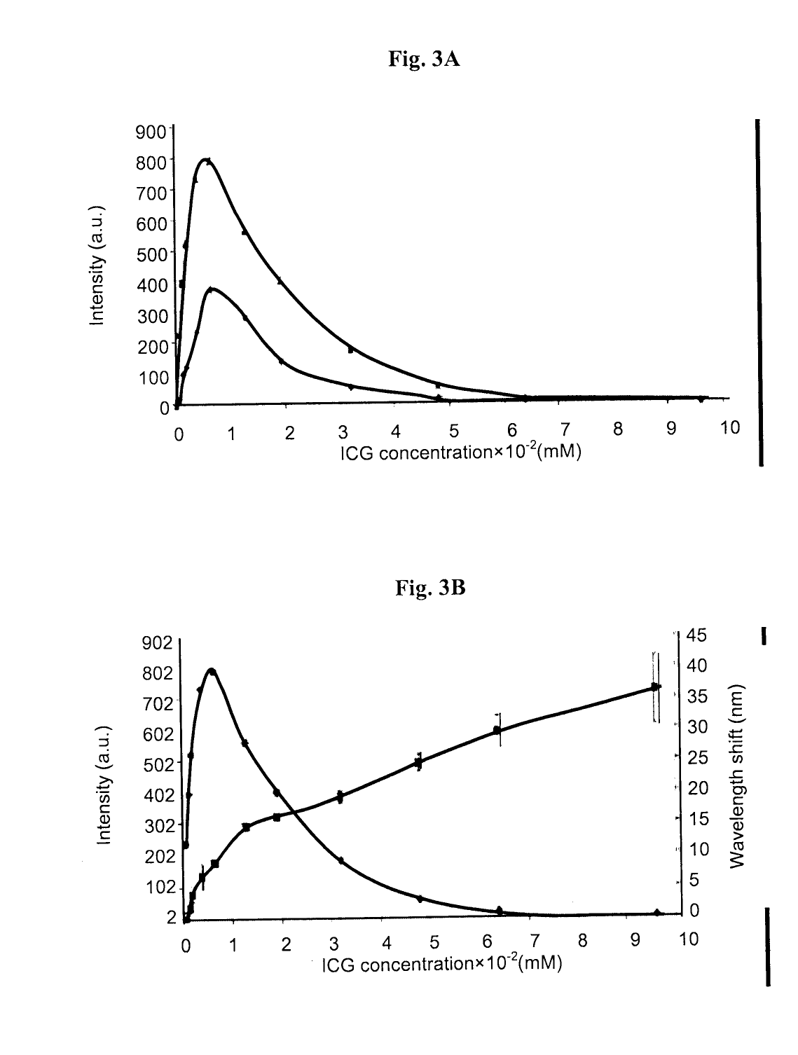

The fluorescence spectra of the ICG-adsorbed liposomes prepared in Example 1 were evaluated in comparison to those of ICG in aqueous solution. It should be noted that because of a red shift that occurs in the systems containing the liposomes, as discussed hereinbelow, the emission intensities will be presented at the wavelengths in which the maximum of the peak was observed. FIG. 3A shows the fluorescence intensities of ICG solutions and for ICG bound to liposomes. As shown, the fluorescence intensity increases with the increase of ICG concentration, reaches a maximum at 6.4×10⁻³ mM, and is followed by a gradual decrease. The concentration at which the maximum is observed is close to that previously reported (Saxena et al., 2003) (2.6×10⁻³ mM) for ICG in aqueous solution. It is clearly seen that the fluorescence intensity is greater for liposomal ICG throughout nearly the entire concentration range studied.

The decrease in fluorescence intensity at high ICG concentrations, both in solution and in liposomal dispersion, was explained by quenching due to formation of aggregates of dye molecules (Saxena et al., 2003). The ICG emission peak in water was observed at 805 nm at concentrations less than 6.4×10⁻³ mM; above that there is a shift up to 814 nm. Those results correlate well with data previously reported (Saxena et al., 2003). The dependence of fluorescence in dispersions of liposomes with hound ICG is different: up to 3.2×10⁻³ mM ICG the emission peak occurs at about 820 nm, whereas above this concentration a red shift that is dependent on ICG concentration is observed. As shown in FIG. 3B, increasing the ICG concentration to 9.6×10⁻² mM causes a shift to about 30 nm.

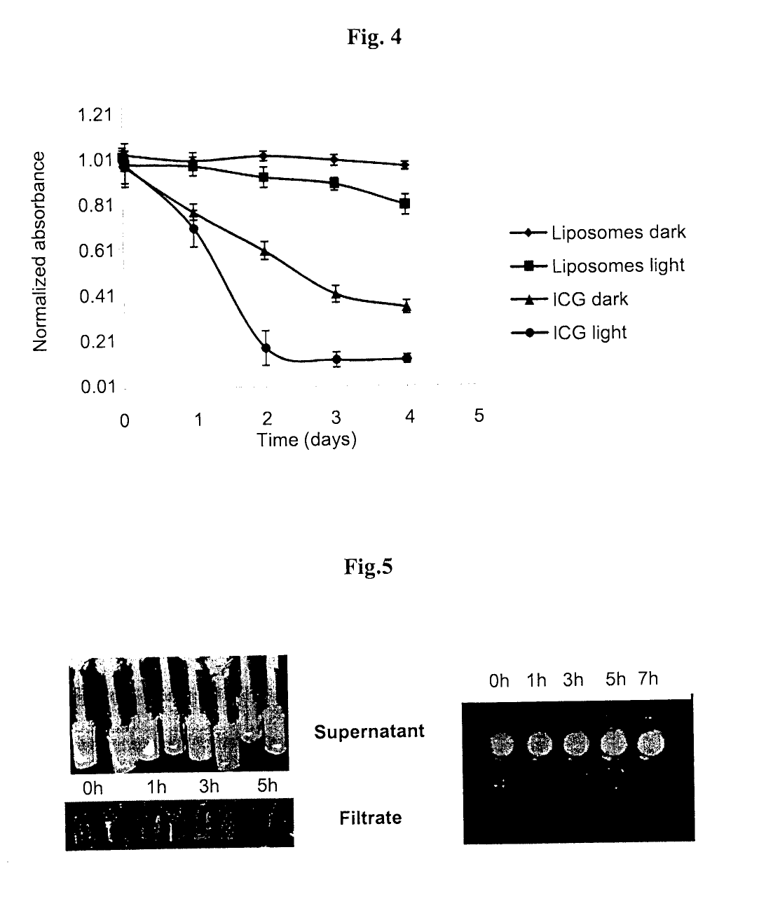

The red shift is accompanied with quenching of fluorescence, i.e., the larger the red shift the lower the fluorescence intensity. Because the fluorescence intensity of ICG-liposomes was greater than that of unbound ICG molecules (aqueous solution), the quantum yield ratio between the two states in which ICG is present was determined, for a concentration of 1.28×10⁻³ mM. As mentioned above, ICG is poorly stable in aqueous solutions due to degradation, and therefore, in order to evaluate the stability of the ICG-adsorbed liposomes, the absorption of this fluorescent probe, when dissolved in an aqueous solution or adsorbed to the liposomes, was measured at light and at dark over time. FIG. 4 shows that while dissolved ICG absorption decreases over time both in light and in dark, the absorption of ICG when bound to the liposomes almost does not change, and that while light further decreases dissolved ICG absorption due to photobleaching, the absorption of ICG when bound to the liposomes remains the same, indicating that photobleaching is not significant in this case. Based on these findings, it can be concluded that the stability of ICG when bound to the liposomes is significantly increased compared with that of ICG dissolved in an aqueous solution.

The relative quantum yield was calculated according to the following equation (Fery-Forgues and Lavabre, 1999):

$$\frac{{φ}_{X}}{{φ}_{S}} = ( \frac{{A}_{s}}{{A}_{x}} ) \times ( \frac{{F}_{x}}{{F}_{s}} ) \times {( \frac{{n}_{x}}{{n}_{s}} )}^{2}$$

where φ is the quantum yield, F is the area under the emission peak (expressed in number of photons), A is absorbance at the excitation wavelength, and n is the refractive index of the solvents. The subscript x denotes the respective values of the sample (liposome-ICG), and s denotes standard (free ICG in buffer). As found, the quantum yield of liposomal ICG is more than three times greater than that of free ICG in buffer solution.

### Example 3

### Stability of ICG-Adsorbed Liposomes in Human Colon Fluid

The stability of the ICG-adsorbed liposomes (nanoprobes) in physiological conditions was tested by incubating the nanoparticles in human colon fluid for 7 hrs. As found by fluorescence measurements, although the ICG is not covalently bound to the liposome, it remained bound to the liposomes during at least 7 hrs, as shown in FIG. 5, indicating that the nanoprobes did not disintegrate in the colon fluid.

### Example 4

### IgG Binding to ICG-Adsorbed Liposomes

Active targeting can be achieved by conjugation of targeting molecules to liposomes, which specifically bind to an antigen or receptor that is overexpressed on the tumor cell surface. In the present study, human IgG was used as a model recognition ligand for evaluating its binding to the ICG-adsorbed liposomes.

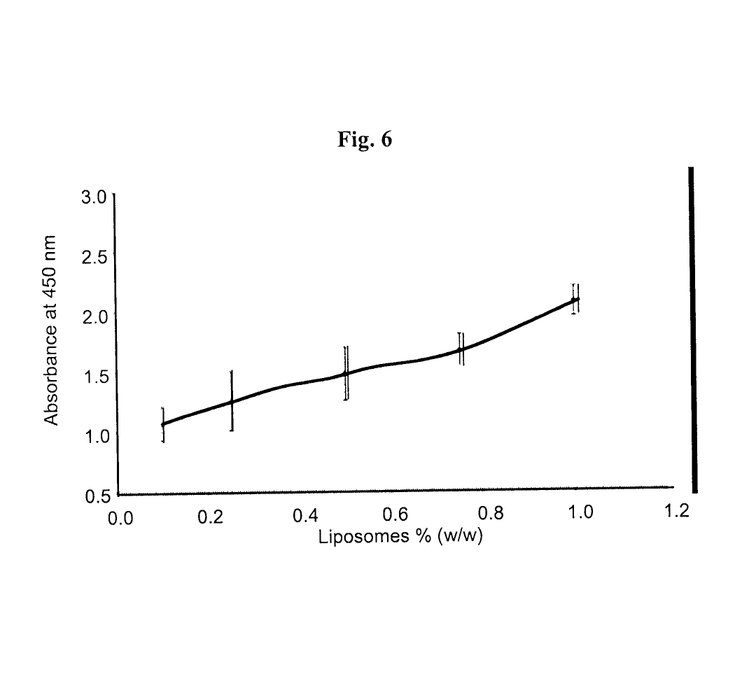

As discussed by Torchilin and Klibanov (1981), binding of proteins Onto the liposome surface can be achieved by simple adsorption. Using this approach, we incubated preformed liposomes with solutions of IgG, followed by removal of the unbound IgG by ultrafiltration. The evaluation of IgG binding to the liposomes was performed by an immunoassay (ELISA). As shown in FIG. 6, the immunoassay results show an increase in the HRP related signal with an increase in liposome-to-IgG ratio. Nonspecific binding to the ELISA plate did not occur in this experiment, because there was no HRP signal in the samples that did not contain IgG.

### Example 5

### Purification and Validation of EGF-FITC Activity

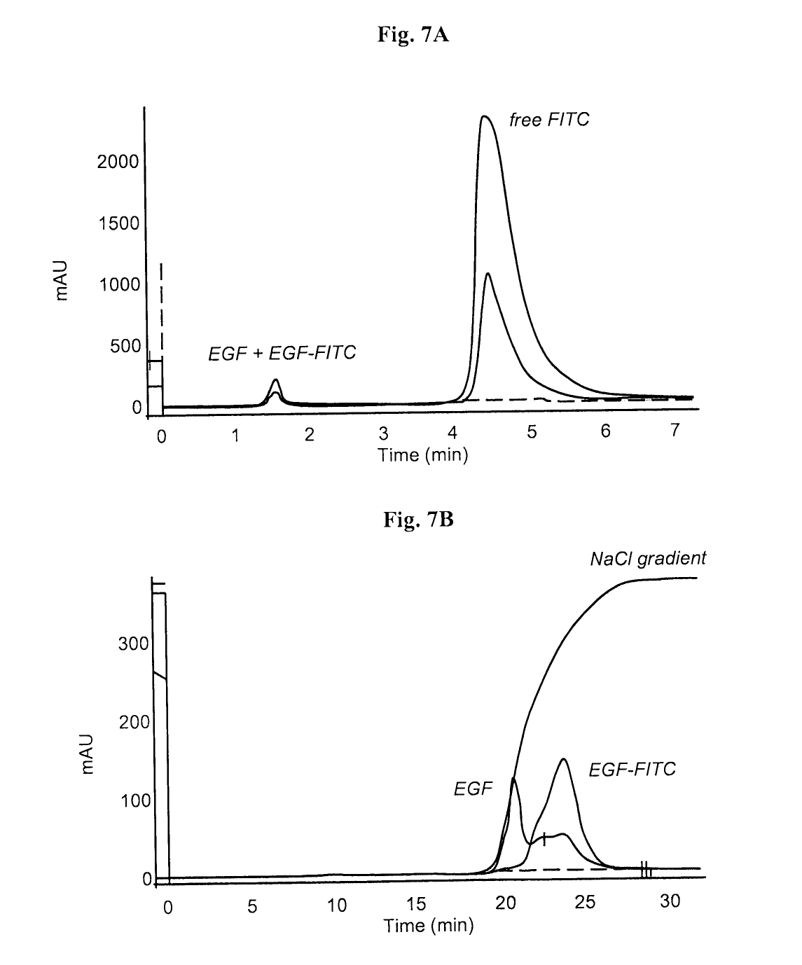

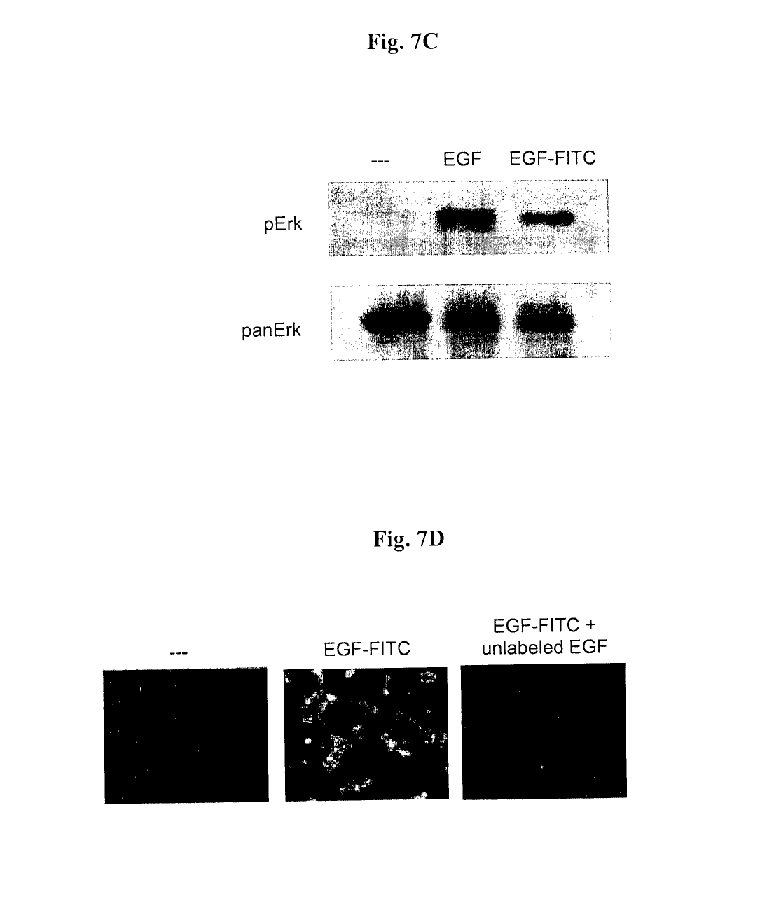

In order to study the binding of cetuximab-labeled ICG-adsorbed liposomes to cell overexpressing EGFR we prepared FITC-labeled EGF (EGF-FITC) to be used as positive control. EGF-FITC was purified from excess FITC and unlabeled FOP via desalting and ion exchange purification steps. Following desalting, the fraction containing EGF and EGF-FITC was collected (FIG. 7A) and further processed to separate labeled from unlabeled EGF. At the ion exchange column, the fraction exhibiting optical absorption at 280 nm (for protein) and 495 nm (for FITC) was collected and referred to as EGF-FITC (FIG. 7B). Upon purification of EGF-FITC, the bioactivity of the labeled protein was evaluated. As presented in FIG. 7C, EGF-FITC was able to induce Erk phosphorylation, an EGFR downstream signaling molecule, similar to that of native EGF. Furthermore, incubation of A431 cells with EGF-FITC resulted in highly specific labeling, which was significantly reduced when incubating the cells with an excess of unlabeled native EGF (FIG. 7D). Following validation of the bioactivity of the labeled protein, EGF-FITC was used in the next experiments as a positive control.

### Example 6

### Specific Binding of Cetuximab-Labeled ICG-Adsorbed Liposomes Evaluated by Confocal Microscopy

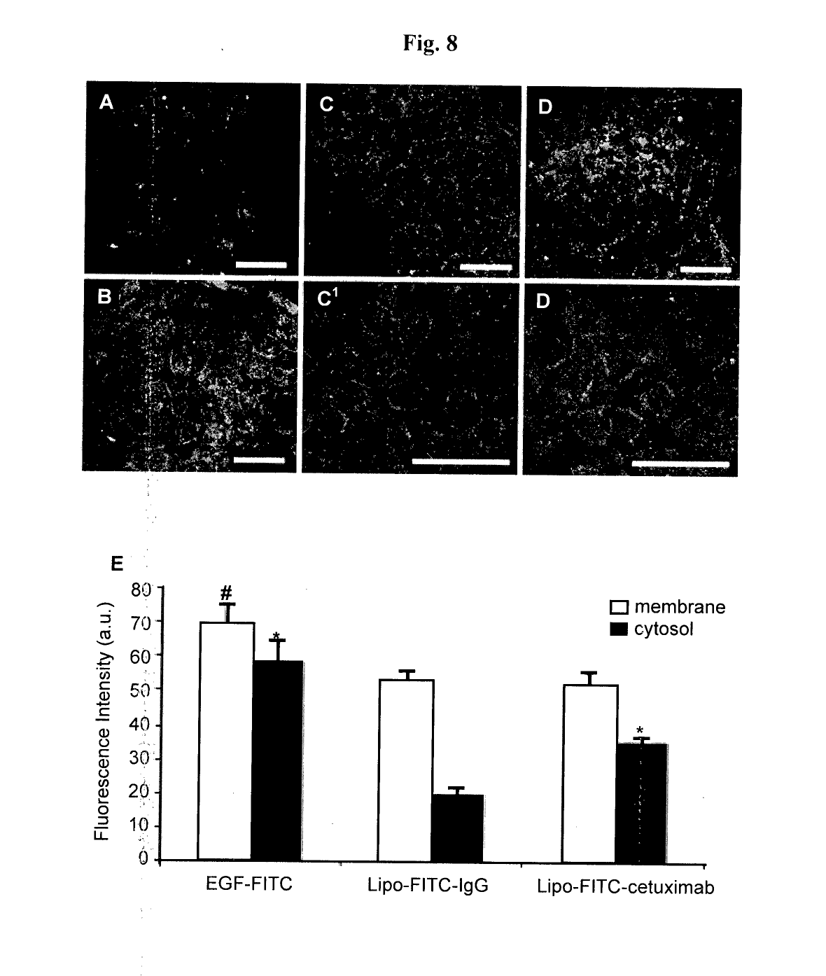

In order to evaluate whether an antibody adsorbed to a liposome preserves its specific recognition ability, we performed binding experiments between the cetuximab (monoclonal antibody to EGFR)-labeled ICG-adsorbed liposomes and A431 colon carcinoma cells overexpressing EGFR. The specificity of cetuximab-labeled liposomes in comparison to nonspecific IgG antibody-labeled liposomes, liposomes only, and fluorescent EGF, was investigated by confocal microscopy, and the results are shown in FIG. 8 (panels A-E).

The nonspecific labeling of the cultures by nonlabeled liposomes was very low (panel A), reflecting mainly the autofluorescence of the cells. The binding of IgG-labeled liposomes was eightfold greater than that of nonlabeled liposomes to the cell culture (panel C and data not shown), most probably reflecting non-EGFR-mediated cell association. The binding of cetuximab-labeled liposomes to the cell culture was about 10-fold greater than that of non-labeled liposomes (panel D and data not shown). Interestingly, careful visualization of the micrographs at high magnification indicates that whereas IgG-labeled liposomes are mainly associated with cell plasma membrane (panel C′), cetuximab-labeled liposomes are both associated with cell plasma membrane and internalized into the cells (panel D′). Quantization of liposome distribution between the plasma membrane and intracellular compartment (panel E) clearly indicates that a greater fraction of cetuximab-labeled, compared with IgG-labeled, liposomes is found in intracellular compartments.

It should be noted that A431 and IEC-6 cultures treated with these liposomes were alive for several days, indicating lack of toxicity of these preparations (data not shown).

### Example 7

### Specific Binding of Cetuximab-Labeled ICG-Adsorbed Liposomes Evaluated by NIR Imaging of Living Cells

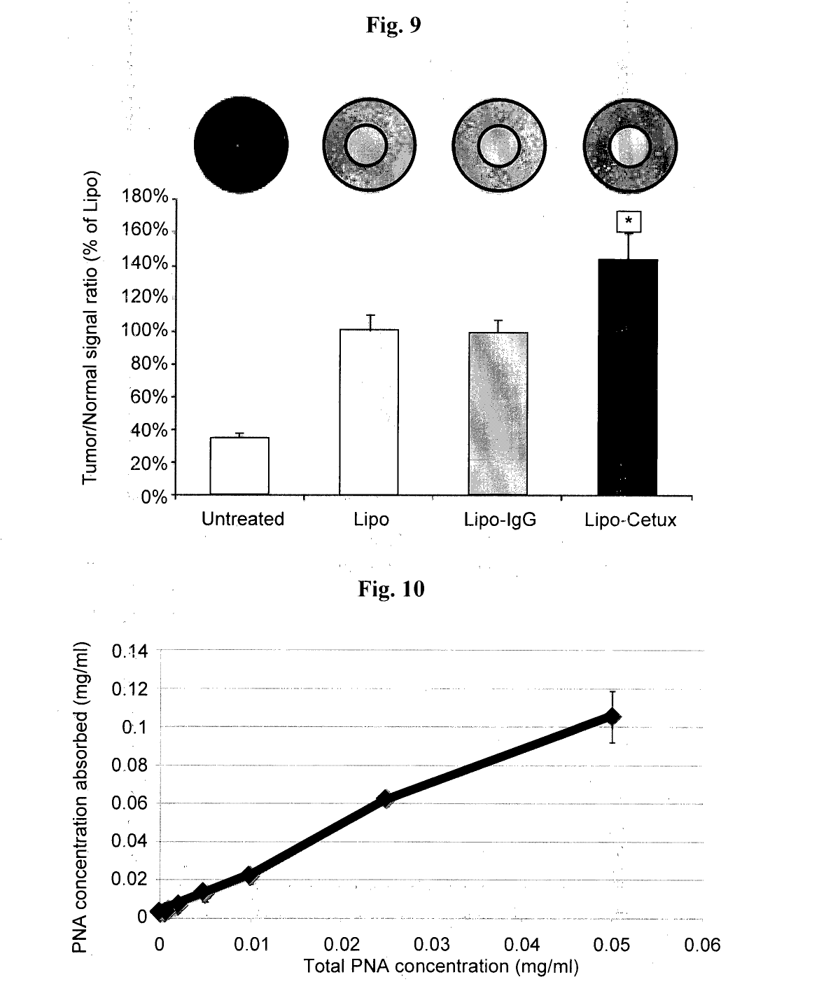

In another approach to evaluating the specific binding of cetuximab-labeled ICG-adsorbed liposomes, we took advantage of a focal in vitro model in which A431 cells are plated in the center and IEC-6 colon cells (normal enterocytes expressing physiological EGFR levels) surround the tumor cells (FIG. 9). Exposure of this tumorlike in vitro model system to cetuximab-labeled liposomes resulted in a specific NIR imaging signal detected with the tumor cells but not with the surrounding normal colorectal cells. As shown in FIG. 9, the binding of cetuximab-targeted fluorescent liposomes to A431 compared with IEC-6 cells was larger by a factor of 3.5, and was larger than nonspecific binding of liposomes without the targeting moiety (cetuximab) by 40%, whereas IgG-labeled liposomes also caused relatively high background labeling. As expected, the control untreated cultures emitted very low NIR autofluorescence signal.

### Example 8

### PNA-FITC Binding to ICG-Adsorbed Liposomes

PNA solution (0.01-0.1 mg/ml in phosphate buffer 0.1 M) was added 1:1 to 1% w/w ICG-adsorbed liposomal dispersion. The solution was incubated for 24 hrs at 5° C. and was then filtered by filtration tube of 300 kDa for 10 min at 40 rpm, and both the filtrate and supernatant were analyzed at 480 nm by fluoresce plate reader. FIG. 10 shows the calculated quantity of PNA-FITC adsorbed to the ICG-adsorbed liposomes as a function of PNA concentration, using fluorescence.

### Example 9

### Specific Recognition by PNA-Labeled ICG-Adsorbed Liposomes

ICG was incubated with 1% liposomal dispersion for 24 hrs at 5° C. under mild shaking. The final concentration of ICG was 3.2×10⁻⁵ M. PNA solution (0.1 mg/ml in phosphate biffer 0.1 M) was added 1:1 to 1% w/w liposomal dispersion, which was then filtered by filtration tube of 300 kDa for 10 min at 40 rpm 3 times to remove the free protein, while 1 ml of water was added at every centrifuge stage.

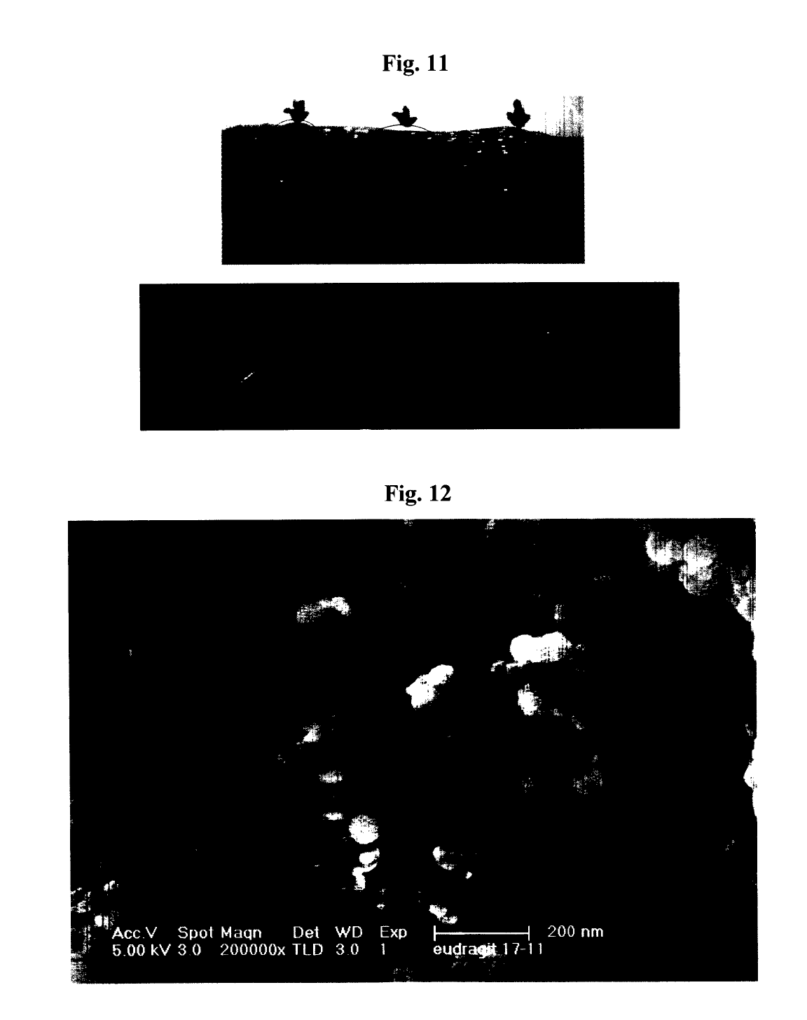

For analysis of specific recognition by PNA, 0.05% PNA-FITC-labeled ICG-adsorbed liposomal dispersion (0.05 mg/ml) was incubated for 40 min with DMH-treated rat colon where tumors were clearly visually seen. The colon was washed twice and was then scanned with fixed intensity at Ex 785 nm and Em 800 nm using Odyssey NIR laser scanner (Li-Cor, Lincoln, Nebr., USA). The images obtained were quantified using the Li-Cur imaging program provided with the scanner. Well identified polyps were successfully signed by phospholipid nanoparticles-PNA-ICG, the signal to noise ratio was 7.03±1.37 relatively to surrounding tissue, as shown in FIG. 11.

### Example 10

### Preparation of Freeze Dried Liposomes Powder

Liposomal dispersions were prepared as described in Examples 5 and 9, and were then mixed with an equal volume of 10 w/w % of sucrose solution (in phosphate buffer 7, 5 mM), wherein the maximal liposome concentration in the obtained formulations was 2.5 w/w %.

The formulations were frozen at −75° C. for 12 hrs and were then lyophilized at −50° C. for an additional 12 hrs. The temperature was elevated to −5° C. for 1.5 hrs, and was then elevated to 5° C. for an additional 1.5 hrs. The freeze dried nanoparticles retained their original size and fluorescence after reconstitution in distilled water.

### Example 11

### Preparation of ICG-Adsorbed Polycationic Nanoparticles

Dispersions of nanoparticles composed of the cationic polymer Eudragit RS 100, a copolymer of acrylate and methacrylates with a quaternary ammonium group, were obtained by precipitation-solvent evaporation. Preliminary results of DLS measurements for the dispersion that contains 2% Eudragit RS and 4% Pluronic F-68 showed an average size of 100 nm (by volume distribution). SEM image of such nanoparticles, which are spherical, is shown in FIG. 12.

By zeta-potential measurements it was found that the nanoparticles are indeed positively charged, having a zeta potential of +16.34±1 mV, that should enable adsorption of the ICG molecules. The zeta potential measured for the ICG-labeled nanoparticles (final ICG concentration of 10⁻⁵M) is 18.42±3.

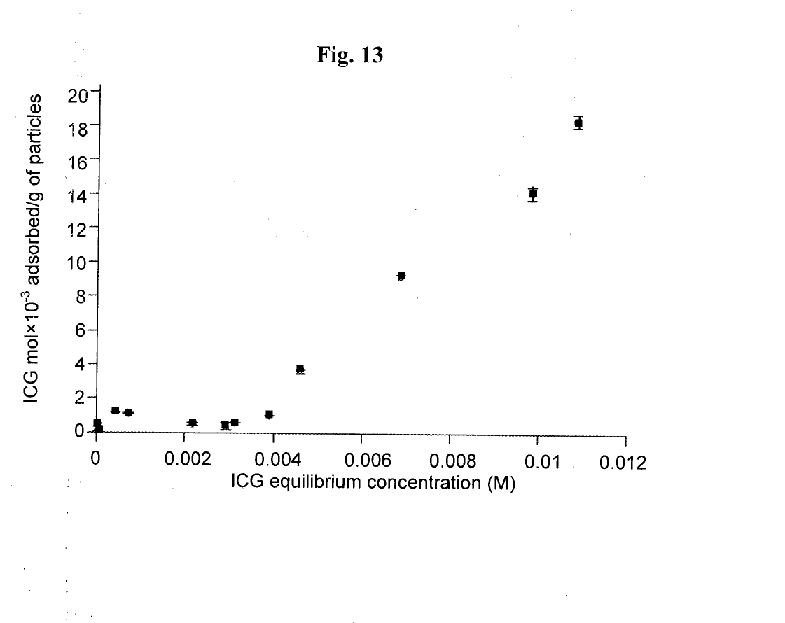

The ICG adsorption was evaluated by incubating the nanoparticles dispersion with a solution of ICG at various concentrations. The adsorbed amount was calculated by measuring the absorbance of the solution obtained after filtration of the dispersion. Typically, it was found that at low ICG concentrations the filtrate did not contain the ICG, while the retained particles had a green color indicating that the ICG was adsorbed onto the nanoparticles. FIG. 13 shows the adsorption isotherm of adsorbed ICG per gram of nanoparticles as a function of free ICG at equilibrium. As shown, the amount of ICG that adsorbed to the particles increases as the equilibrium concentration increases at the higher concentration (0.004 M).

It should be noted that at high ICG concentrations, aggregation of the nanoparticles occurred. The maximal concentration of ICG at which there was no aggregation was 10⁻⁵ M. At this concentration the amount of adsorbed ICG molecules is approximately 10⁴ ICG molecules per nanoparticle (Yuan et al., 2004).

### Example 12

### Fluorescence of the ICG-Adsorbed Polycationic Nanoparticles

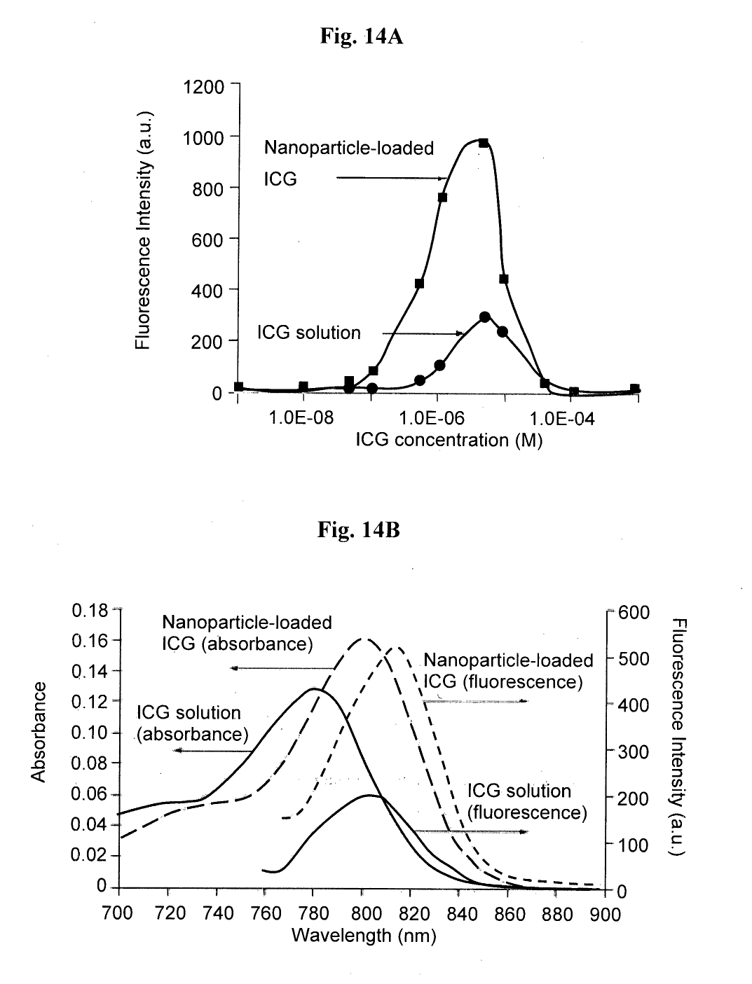

In order to evaluate the fluorescence intensity after the ICG attachment to the polycationic nanoparticles, the fluorescence of the particles in dispersions obtained after removal of the filtrate was measured for particles prepared at ICG concentrations less than 10⁻³ M. Such typical NIR fluorescence, which proves that the adsorbed ICG molecules retain their optical properties, is shown in FIG. 14A, with comparison to that of ICG in aqueous solution. The ICG concentrations that were used enabled high emission intensity without reaching the self-quenching concentration (10⁻⁴ M). FIG. 14A illustrates the improvement in the fluorescence intensity in the particles compared with ICG aqueous solution, and FIG. 14B shows the absorption emission profile of ICG-loaded nanoparticles compared with ICG aqueous solution both at a concentration of 10⁻⁶ M.

Since the emission of the particles was higher than the ICG aqueous solution emission, the quantum yield ratio between the particles and the free ICG was calculated as described in Example 2, and as found, the quantum yield of ICG-loaded nanoparticles is 5.3-times greater than that of free ICG in aqueous solution.

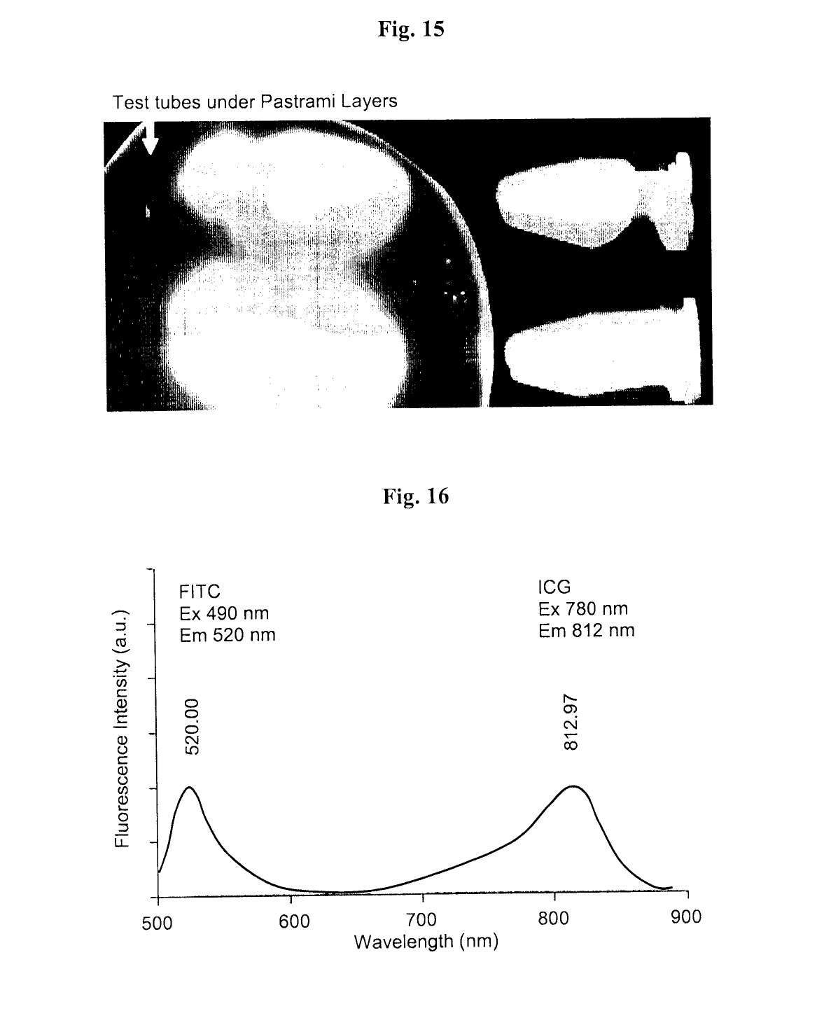

The possible applicability of the NIR nanoparticle for remote imaging based on NIR fluorescence was further evaluated by imaging the ICG through a layer mimicking human tissue. From the imaging experiments it was found that the fluorescence from the test tube containing the ICG-loaded nanoparticles could be visualized even when it is below 1.6 cm of a tissue mimicking solid (FIG. 15), indicating that the nanoparticles can be used for remote fluorescence sensing.

### Example 13

### IgG Binding to ICG-Adsorbed Polycationic Nanoparticles

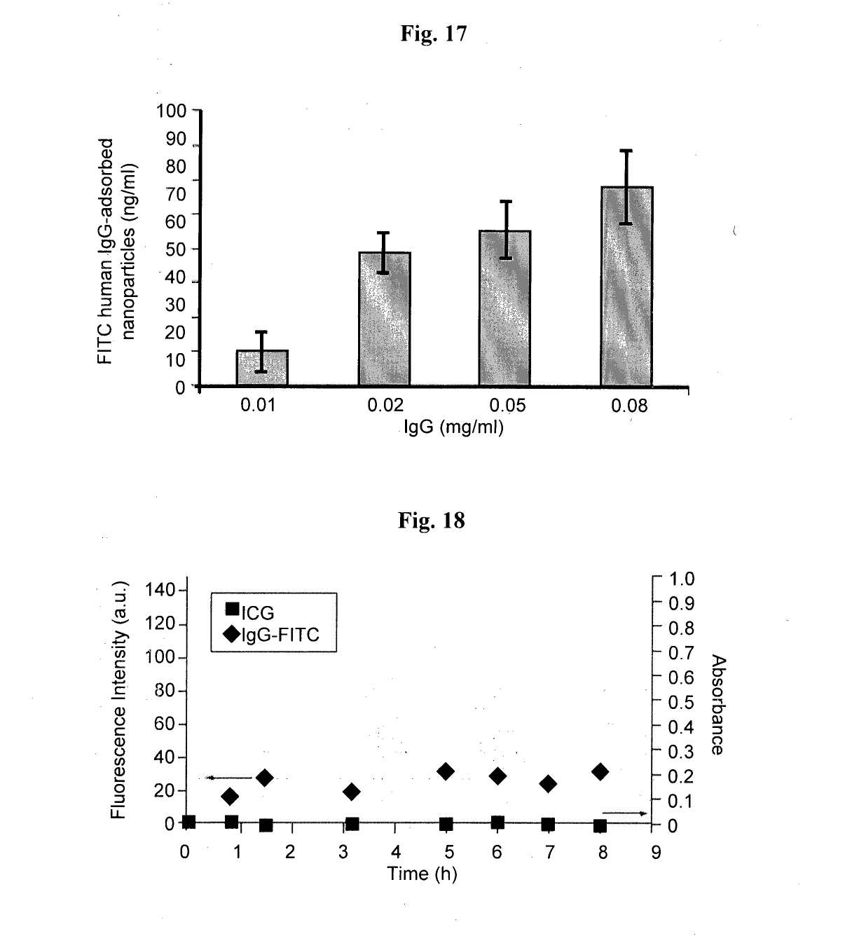

In this study, the possibility to utilize the polycationic nanoparticles for biological imaging and binding of targeting molecule was evaluated. As a model targeting molecule, human IgG was applied, again by non-covalent attachment to the ICG-adsorbed polymeric particles. These experiments were conducted while using FITC-labeled IgG, which enabled a qualitative evaluation of the binding by fluorescence measurements of the particles. The adsorption of IgG molecules to the particles was evaluated by an ELISA-immunoperoxidase assay. The emissions of these particles were measured with excitation at wavelengths that are suitable for the two dyes: FITC-IgG and ICG (at 490 and 780 nm, respectively). As presented in FIG. 16, emissions of both FITC and ICG were obtained at 520 and 812 nm, respectively, meaning that IgG-FITC molecules were attached to the ICG-labeled nanoparticles, again by noncovalent bonding. Following these preliminary fluorescence results, ELISA experiments were performed in order to evaluate the binding of IgG molecules adsorbed to the particles. The ELISA experiments tested the attachment of the IgG-labeled nanoparticles to the walls of polystyrene microtiter wells, which are pre-coated with anti-human IgG antibodies. The experiments were conducted with a constant quantity of particles and the adsorbed quantity of IgG was varied. From the ELISA results shown in FIG. 17, it can be seen that as the concentration of adsorbed IgG per particle is increased, the quantity of IgG that is hound to the wells increases. It should be noted that all the ELISA results are presented with proper controls and with subtraction of the background reading, which may result from binding of nanoparticles only.

### Example 14

### Stability of IgG-Labeled ICG-Adsorbed Polycationic Nanoparticles

Since the ICG is non-covalently hound to the Eudragit RS 100 particles, in order to utilize these particles in a biological medium, it was essential to evaluate whether the ICG/IgG-FITC remain bound to the particles in physiological conditions. Therefore, the IgG-FITC-labeled ICG-adsorbed nanoparticle dispersion was incubated with intestinal fluid (pH 16.8) for 8 hrs. As shown in FIG. 18, the particles were physically stable and the ICG/IgG-FITC did not dissociate from the particles. The total intensity of IgG-FITC was three-times higher than the intensity obtained from the free IgG-FITC at time=0. It should be noted that all the fluorescence results are presented with subtraction of the free IgG at time=0.

### Example 15

### Preparation of EGF-Labeled ICG-Adsorbed Polycationic Nanoparticles

In this experiment, ICG-adsorbed polycation nanoparticle dispersion prepared was mixed 1/1 v/v with a diluted EGF solution (EGF stock solution was prepared by 50 times dilution of 500 μg of EGF in distilled water, 0.1-10 μg/ml) for 24 hrs, and was then filtered by filtration tube of 300 kDa for 15 min at 60 rpm 3 times to remove free EGF, while 1 ml water was added at every centrifuge stage.

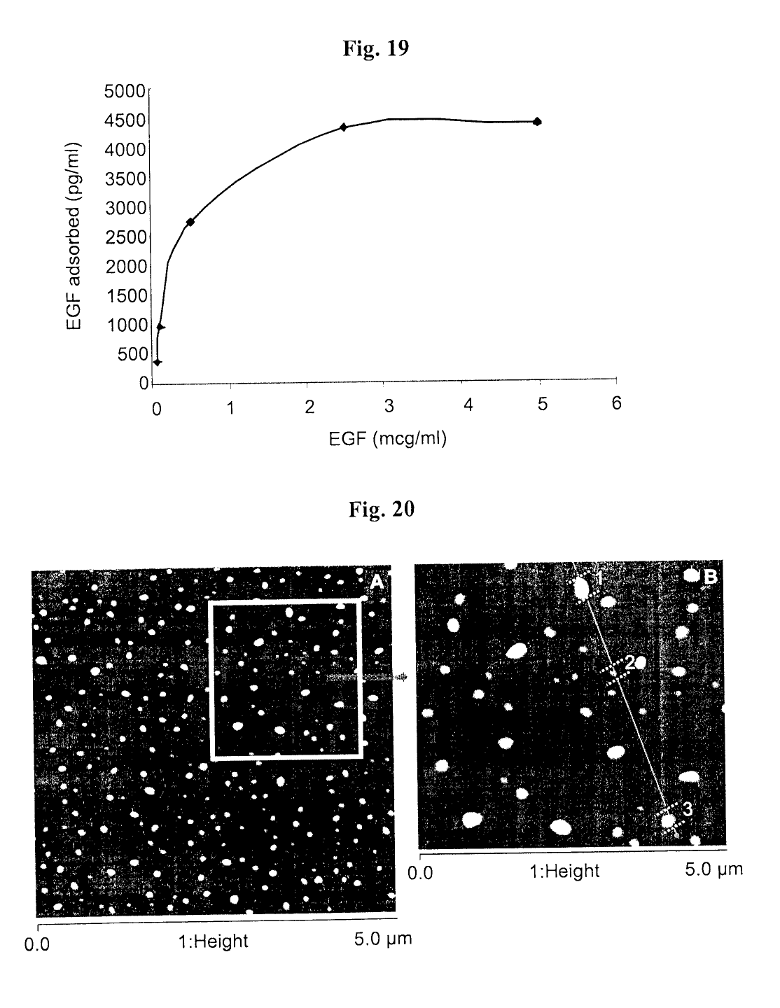

In order to quantify the amount of EGF adsorbed on the nanoparticles, ELISA experiments were performed with the Human EGF ELISA Development Kit (900-K05, Peprotech). All the recommended materials and solutions were prepared according to the ELISA protocol, and the ELISA plate was read by ELISA reader at wavelength of 405 nm and correction at 650 am. As shown in FIG. 19, the absorbance measured for labeled particles was significantly higher than that measured for unlabeled particles, indicating stable non-covalent interactions between the ICG-adsorbed polycation nanoparticles and the EGF adsorbed thereto.

### Example 16

### Preparation of ICG-Adsorbed Eudragit RS-Casein Polycationic Nanoparticles

Sodium caseinate-adsorbed polycation nanoparticles (Eudragit-casein nanoparticles) were prepared by the precipitation and solvent evaporation method according to the following procedure: the cationic polymer Eudragit RS was dissolved in acetone (10 wt %) and stirred by magnetic stirrer for at least 20 min. TDW, filtered by 0.22 μm syringe filter, and acetone solution were poured simultaneously into a vial while stirring with magnetic stirrer. The acetone/water solutions ratio was 20:80, respectively. The acetone was removed by evaporator at room temperature and replaced with filtered TDW to get a final dispersion concentration of 2 wt %. Next, 0.1 gr of sodium caseinate was dissolved in 45 gr 10 mM PBS solution by stirring with magnetic stirrer for 30 min to get a micellar solution (0.22 wt %). 5 gr of the polycation nanoparticles dispersion was added to the micellar solution while stirring with magnetic stirrer for 24 hrs at room temperature.

By using the above procedure, size measurements (DLS) of the polymeric nanoparticles after sodium caseinate adsorption showed an average diameter of 200 nm which agreed well with AFM image (FIG. 20) and zeta potential of −29 mV. The measurements were performed by Zetasizer Nano-S (Malvern) and atomic force microscope (Dimension 3100 Nanoscope V, Veeco).

ICG stock solution of 10⁻³ M was prepared by dissolving 7.75 mg ICG in 10 ml of TDW.

ICG-adsorbed Eudragit-casein nanoparticles were prepared by adding 300 μl of ICG stock solution to 2.7 ml of the Eudragit-casein nanoparticle dispersion, followed by mild shaking for 2 hrs. The particles retained their original size and zeta potential.

The ICG-adsorbed Eudragit-casein nanoparticle dispersion was then filtered by centrifugation in a 300 kDa filtration tube (VS0241 VIVA SPIN) for 5 min at 1000 rpm (Centrifuge CN-2200 MRC) in order to remove the free ICG. As found, the filtrate did not contain ICG, while the retained particles had green color indicating that the ICG was attached to the nanoparticles by non-covalent interactions. The emission of the retained particles was measured by Cary Eclipse fluorimeter (excitation at 800 nm, emission at 830 nm).

### Example 17

### Stability of ICG-Adsorbed Eudragit RS-Casein Polycationic Nanoparticles in Human Colon Fluid

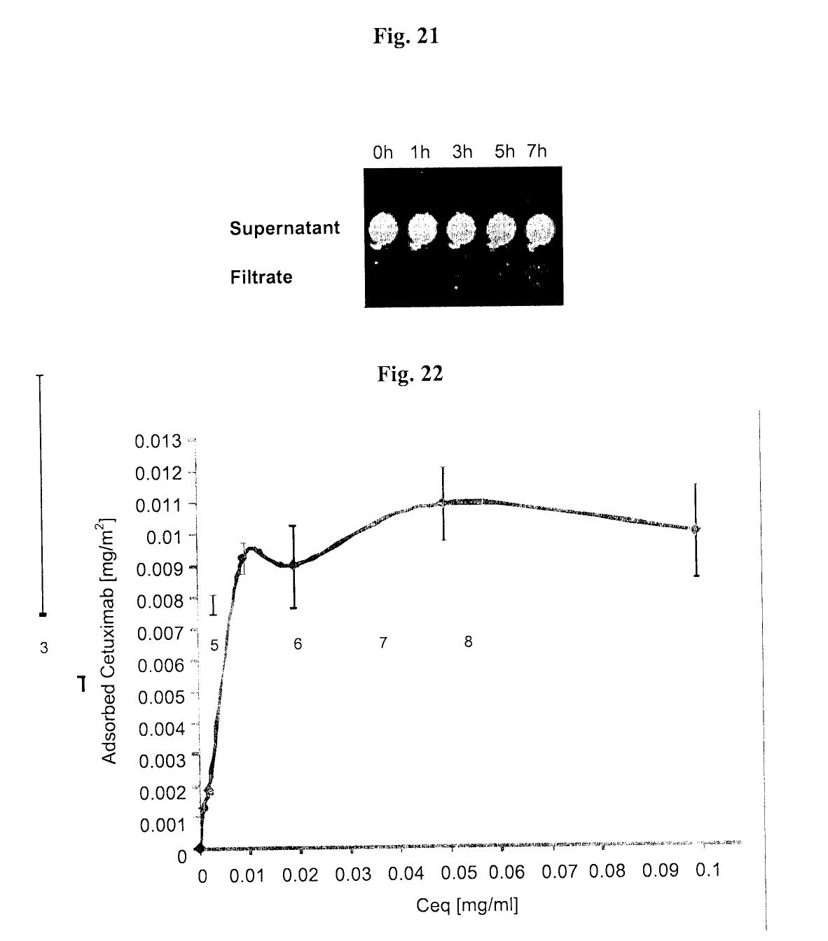

ICG-adsorbed Eudragit-casein nanoparticle dispersion (ICG concentration 10⁻⁵ M) was diluted 1:18 in fresh colon fluid which was first filtrated by 5μ and 0.45μ pore size membranes. The samples were incubated at 37° C. up to 7 hrs during mild shaking. The samples for each time point were filtered by filtration tube of 300 kDa for 10 min at 40 rpm. The filtrates and the supernatant were screened by NIR laser scanner (Li-Cor, Lincoln, Nebr., USA) (FIG. 21).

### Example 18

### Preparation of Cetuximab-Labeled ICG-Adsorbed Eudragit-Casein Polycationic Nanoparticles

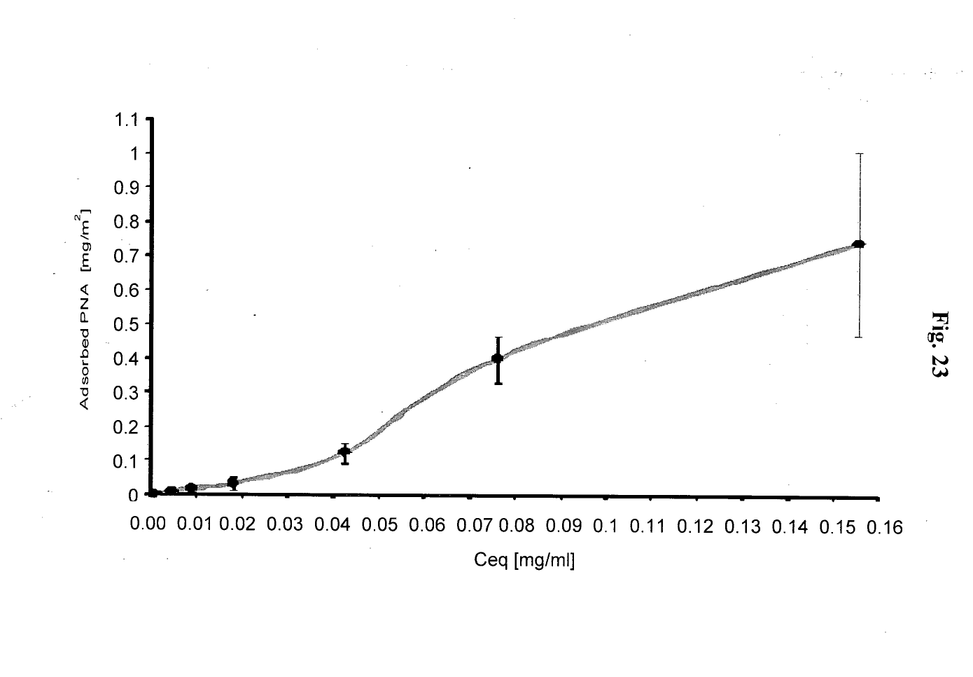

Cetuximab solution (1×10⁻⁴-0.01 mg/ml in phosphate buffer 5 mM) was added 1:1 to 0.2 wt % ICG-adsorbed Fudragit-casein nanoparticle dispersion, the reference Eudragit-casein-ICG nanoparticles and cetuximab-labeled ICG-adsorbed Eudragit-casein nanoparticles were diluted in PBS 10 mM. The dispersion was incubated for 24 hrs at 4° C. while mild shaking. Next, the dispersion was filtered by filtration tube (300 kDa) for 5 min at 1000 rpm 3 times in order to remove free cetuximab while at every centrifuge stage, 1 ml of water was added. In order to quantify the amount of antibody adsorbed on the nanoparticles, ELISA experiments were performed with the Human IgG ELISA Development Kit (Human IgG ELISA kit, goat source, E-80G, ENCO). All the recommended materials and solutions were prepared according to the ELISA protocol and the ELISA plate was read by ELISA reader at wavelengths of 450 nm. The absorbance measured for labeled nanoparticles was significantly higher relatively to unlabeled nanoparticles, indicating that cetuximab was attached to the ICG-adsorbed nanoparticles by non covalent interactions, as indicated by the ELISA results shown in FIG. 22.

### Example 19

### Preparation of PNA-FITC-Labeled ICG-Adsorbed Eudragit-Casein Polycationic Nanoparticles

PNA solution (0.01-0.1 mg/ml in phosphate buffer 0.1 M) was added 1:1 to 0.2 wt % ICG-adsorbed Eudragit-casein nanoparticle dispersion, and the solution was incubated for 24 hrs at 4° C. Next, the dispersion was filtered by filtration tube (300 kDa) for 5 min at 1000 rpm. The filtrate and supernatant were analyzed at 480 nm by fluoresce plate reader (FIG. 23).

### Example 20

### In Vivo Specific Recognition in DMH Rats by PNA-FITC-Labeled ICG-Adsorbed Eudragit-Casein Polycationic Nanoparticles

For analysis of specific recognition by PNA, 0.1% ICG-adsorbed Eudragit-casein nanoparticle dispersion containing adsorbed PNA-FITC (0.05 mg/ml) were inserted into DMH-treated rat colon and incubated for 20 min. Next, the colon was washed twice with 10 mM PBS, the rats were sacrificed, and their colon were taken out and scanned with fixed intensity at Ex 785 nm and Em 800 nm, using Odyssey NIR laser scanner (Li-Cor, Lincoln, Nebr., USA). The obtained images were quantified using the Li-Cor imaging program provided with the scanner. Well identified polyps were successfully signed by the nanoparticles, wherein the signal to noise ratio was 4.48±0.46 relatively to surrounding tissue.

### Example 21

### Preparation of Anti-CEA-FITC-Labeled ICG-Adsorbed Eudragit-Casein Polycationic Nanoparticles

In order to evaluate anti-CEA (T84.66) adsorption to ICG-adsorbed Eudragit-casein nanoparticles, FITC was conjugated to anti-CEA by covalently binding the FITC functional group, isothiocyanate, to a primary amine of the anti-CEA. More particularly, FITC was dissolves in anhydrous DMSO to a final concentration of 0.01 gr/ml, and 4 μl of the solution was then added to 500 μl of 0.001 gr/ml anti-CEA solution in PBS. In order to initiate the reaction, 50 μl of 500 mM carbonate buffer (1.7×10⁻⁴ wt % Na₂CO₃, 2.8×10⁻⁴ wt % NaHCO₃; pH 9.5) was added and the final solution was incubated for 2 hrs in 37° C., after which unbound FITC molecules were washed out using ultrafiltration tube (cut off 5000 Da), and 500 μl of 10 mM PBS was added to a final anti-CEA-MC solution concentration of 0.001 gr/ml. In order to validate FITC conjugation, the absorption spectra of the resultant anti-CEA-FITC solution was measured relatively to the same concentration of non conjugated anti-CEA solution (0.001 gr/ml).

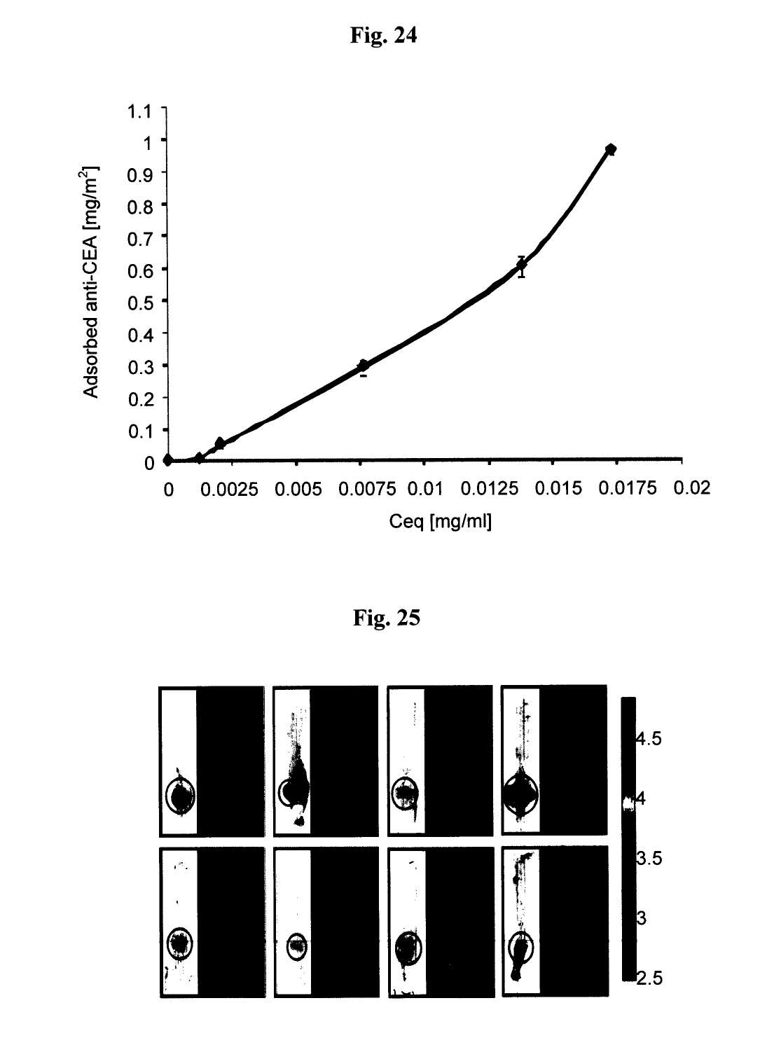

Anti-CEA-FITC solution (0-0.15 mg/ml in PBS 10 mM) was added 1:1 to 0.2 wt % ICG-adsorbed Eudragit-casein nanoparticle dispersion. The dispersion was incubated for 24 hrs at 4° C. while mild shaking. Next, the dispersion was filtered by filtration tube (300 kDa) for 5 min at 1000 rpm 3 times in order to remove free anti-CEA-FITC while at every centrifuge stage, 1 ml of water was added. The filtrate and supernatant were analyzed at 480 nm by fluoresce plate reader (FIG. 24).

### Example 22

### In Vivo Specific Recognition in LS174T Tumor Bearing Mice by ICG-Adsorbed Eudragit-Casein Polycationic Nanoparticles

For analysis of specific recognition by anti-CEA, 0.1% ICG-adsorbed Eudragit-casein nanoparticle dispersion either labeled or not with anti-CEA (0.025 mg/ml) were inserted into the mice colon and incubated for 20 min. Next, the colon was washed with 5 ml 10 mM PBS. After 3 hrs, the mice were sacrificed and their colon were taken out and scanned with fixed intensity at Ex 785 nm and Em 800 nm using Odyssey NIR laser scanner (Li-Cor, Lincoln, Nebr., USA). The obtained images were quantified using the Li-Cor imaging program provided with the scanner.

Well identified LS174T tumors were successfully marked by the anti-CEA-labeled ICG-adsorbed Eudragit-casein nanoparticles, as shown in FIG. 25 (lower panels) (signal to noise ratio was evaluated by image processing software, matlab). ICG-adsorbed Eudragit-casein nanoparticles without anti-CEA label also showed specific recognition to LS 174T tumors, probably due to the acidic environment of the tumor's extracellular fluid (FIG. 25, upper panels).

### REFERENCES

Barenholz Y., Crommelin D. J. A., Liposomes as a pharmaceutical dosage forms. In: Swarbrick B, editor. Encyclopedia of pharmaceutical technology, Vol. 9. New York: Marcel Dekker; 1994. p. 1
Bellare J. R., Davis H. T., Scriven Talmon Y., Controlled environment vitrification system: an improved sample preparation technique. J Electron Microsc Tech, 1988, 10, 87-111
Becker J. C., Muller-Tidow C., Serve H., Domschke W., Pohle T., Role of receptor tyrosine kinases in gastric cancer: new targets for a selective therapy. World J Gastroenterol., 2006, 12(21), 3297-3305
Bogdanova Y. G., Dolzhikova D., Surface and bulk properties of aqueous binary mixtures of Pluronic F-68 and low molecular weight cationic surfactants: 1. surface tension and association in aqueous solutions. Colloid J., 2008, 70(2), 160-165
Boland C. R., Roberts J. A., Quantitation of lectin binding sites in human colon mucins by use of peanut and wheat germ agglutinins. J Histochem Cytochem., 1988, 36(10), 1305-1307
Campbell B. J., Finnie I. A., Hounsell E R, Rhodes J. M., Direct demonstration of increased expression of Thomsen-Friedenreich (TF) antigen in colonic adenocarcinoma and ulcerative colitis mucin and its concealment in normal mucin. J Clin Invest., 1995, 95(2), 571-576
Dabelsteen E., Cell surface carbohydrates as prognostic markers in human carcinomas. J Pathol., 1996, 179(4), 358-369
Damino D., Bernheim-Grosswasser A., Talmon Y., Digital cryogenic transmission electron microscopy: an advanced tool for direct imaging of complex fluids. Colloid Surf A, 2001, 183, 113-122
DeRossi U., Daehne S., Lindrum M., Increased coupling size in J-aggregates through N-nalkyl betaine surfactants. Langmuir, 1996, 12, 1159-1165
Desmettre T., Devoisselle J. M., Mordon S., Fluorescence properties and metabolic features of indocyanine green (ICG) as related to angiography. Surv Ophthalmol, 2000, 45, 15-27
Devoiselle J. M., Soulie-Begue S., Mordon S. R., Desmettre T., Maillols H., Fluorescence properties of indocyanin green: I. In-vitro study with micelles and liposomes. Proc SPIE, 1997, 2980, 530-537
Duffy M. J., van Dalen A., Haglund C., Hansson L., Holinski-Feder E., Klapdor R., Lamers R., Peltomaki P., Sturgeon C., Topolcan O., Tumour markers in colorectal cancer: European Group on Tumour Markers (EGTM) guidelines for clinical use. Eur J Cancer, 2007, 43, 1348-1360
Dzurinko Gurwood A. S., Price J. R., Intravenous and indocyanine green angiography. Optometry, 2004, 75, 743-755
Enders G. H., Colonic polyps. 2009 (http://emedicine.medscape.com/article/172674-overview)
Ferrucci J. T., Virtual colonoscopy for colon cancer screening: further reflections on polyps and politics. Am J Roentgenol, 2003, 181, 795-797
Fery-Forgues S., Lavabre D., Are fluorescence quantum yields so tricky to measure? A demonstration using familiar stationery products. J Chem Educ, 1999, 76, 1260-1264
Ferrucci J. T., Virtual colonoscopy for colon cancer screening: further reflections on polyps and politics. AJR Am J. Roentgenol., 2003, 181(3), 795-797
Gathje J., Steuer R. R., Nicholes K. R., Stability studies on Indocyanine Green dye. J Appl Physiol, 1970, 29, 181-185
Gong H., Kovar J., Little G., Chen H., Olive D. M., In vivo imaging of xenograft tumors using an epidermal growth factor receptor-specific affibody molecule labeled with a near-infrared fluorophore. Neoplasia, 2010, 12, 139-149
Haigler H., Ash J. F., Singer S. J., Cohen S., Visualization by fluorescence of the binding and internalization of epidermal growth factor in human carcinoma cells A-431. Proc Nall Acad Sci USA, 1978, 75, 3317-3321
Horimoto, N. N., Imura, K., Okamoto, H., Dye fluorescence enhancement and quenching by gold nanoparticles: Direct near-field microscopic observation of shape dependence. Chem Phys Lett., 2008, 467(1-3), 105-109
Hu M., Scollard D., Chan C., Chen P., Vallis K., Reilly R. M., Effect of the EGFR density of breast cancer cells on nuclear importation, in vitro cytotoxicity, and tumor and normal-tissue uptake of [111In]DTPA-hEGF. Nucl Med. Biol., 2007, 34(8), 887-896
Jones M. N., Liposomal antibacterial, antifungal and antiviral agents. In: Nejan D, editor. Methods in enzymology, 2005, Vol. 391, p. 218. San Diego: Elsevier-Academic
Kirchherr A. K., Briel A., Mader K., Stabilization of Indocyanine Green by encapsulation within micellar systems. Mol Pharmaceut, 2009, 6, 480-491
Koyama Y., Barrett T., Hama Y., Ravizzini G., Choyke P. L., Kobayashi H., In vivo molecular imaging to diagnose and subtype tumors through receptor-targeted optically labeled monoclonal antibodies. Neoplasia, 2007, 9(12), 1021-1029
Kriete A., Papazoglou E., Edrissi B., Pais H., Pourrezaei K., Automated quantification of quantum-dot-labelled epidermal growth factor receptor internalization via multiscale image segmentation. J Microsc., 2006, 222(Pt 1), 22-27
Lai S. K., Wang Y. Y., Hanes J., Mucus-penetrating nanoparticles for drug and gene delivery to mucosal tissues. Adv Drug Deliv Rev., 2009, 61(2), 158-171
Lecht S., Arien-Zakay H., Wagenstein Y., Inoue S., Marcinkiewicz C., Lelkes P. I., Lazarovici P., Transient signaling of Erk1/2, Akt and PLCgamma induced by nerve growth factor in brain capillary endothelial cells. Vascul Pharmacol, 2010, 53, 107-114
Leung T. S., Tachtsidis I., Tisdall M., Smith M., Delpy D. T., Elwell C. E., Theoretical investigation of measuring cerebral blood flow in the adult human head using bolus Indocyanine Green injection and near-infrared spectroscopy. Appl Opt., 2007, 46(10), 1604-1614
Maia M., Margalit E., Lakhanpal R., Tso M. O., Grebe R., Torres G., Au Fong K. G., Farah M. E., Fujii G. Y., Weiland J., de juan E Jr., D'Anna S. A., 1-Iumayun M. S., Effects of intravitreal indocyanine green injection in rabbits. Retina., 2004, 24(1), 69-79
Mamot C., Drummond D. C., Noble C. O., Kallab V., Guo Z., Hong K., Kirpotin D. B., Park J. W., Epidermal growth factor receptor-targeted immunoliposomes significantly enhance the efficacy of multiple anticancer drugs in vivo. Cancer Res, 2005, 65, 11631-11638
Mamot C., Ritschard R., Kung W., Park J. W., Hemnann R., Rochlitz C. F., EGFR-targeted immunoliposomes derived from the monoclonal antibody EMD72000 mediate specific and efficient drug delivery to a variety of colorectal cancer cells. J Drug Target, 2006, 14(4), 215-223
Ogawa M., Kosaka N., Choyke P. L., Kobayashi H., In vivo Molecular Imaging of Cancer with a Quenching Near-Infrared Fluorescent Probe Using Conjugates of Monoclonal Antibodies and Indocyanine Green. Cancer Res., 2009, 69(4), 1268-1272
Philip R., Penzkofer A., Baumler W., Szeimies R. M., Abels C., Absorption and fluorescence spectroscopic investigation of indocyanine green. J Photoch Photobio A., 1996, 96(1-3), 137-148
Proulx S. T., Luciani P., Derzsi S., Rinderknecht M., Mumprecht V., Leroux J. C., Detmar M., Quantitative imaging of lymphatic function with liposomal indocyanine green. Cancer Res, 2010, 70, 7053-7062
Sakka S. G., Assessing liver function. Curr Opin Crit Care, 2007, 13, 207-214
Sakuma S., Yano T., Masaoka Y., Kataoka M., Hiwatari K., Tachikawa H., Shoji, Y., Kimura R., Ma, H., Yang, Z., Tang L., Hoffman, R. M., Yamashita S., In vitro/in vivo biorecognition of lectin-immobilized fluorescent nanospheres for human colorectal cancer cells. J Control Release., 2009, 134(1), 2-10
Sandanaraj B. S., Gremlich H. U., Kneuer R., Dawson J., Wacha S., Fluorescent nanoprobes as a biomarker for increased vascular permeability: implications in diagnosis and treatment of cancer and inflammation. Bioconjug Chem, 2010, 21, 93-101
Saxena V., Sadoqi M., Shao J., Degradation kinetics of indocyanine green in aqueous solution. J Pharm Sci., 2003, 92(10), 2090-2097
Saxena V., Sadoqi M. Shao J., Indocyanine green-loaded biodegradable nanoparticles: preparation, physicochemical characterization and in vitro release. Int J Pharm, 2004, 278, 293-301
Seferis J. C., Refractive indices of polymers. In: Polymer Handbook, 4^{th} Edition. Brandrup J, Immergut E H, Grulke E S (Eds). Wiley, NY, USA, VI/571 (1999)
Sevick-Muraca E. M., Lopez G., Reynolds J. S., Troy T. L., Hutchinson C. L., Fluorescence and absorption contrast mechanisms for biomedical optical imaging using frequency-domain techniques. Photochem Photobiol., 1997, 66(1), 55-64
Sidorowicz, A., Po, A., Dobryszycki. P., Spectral properties of 3,3′-diethyloxadicarbocyanine included in phospholipid liposomes. Journal of Photochemistry and Photobiology B: Biology, 1997, 38(1), 94-97
Sidorovicz A., Mora C., Jblonka S., Pola A., Modrzycka T., Mosiadz D., et al. Spectral properties of two betaine-type cyanine dyes in surfactant micelles and in the presence of phospholipids. J Mol Struct, 2005, 744, 711-716
Singh R., Campbell B. J., Yu L. G., Fernig D. G., Milton J. D., Goodlad R. A., FitzGerald A. J., Rhodes J. M., Cell surface-expressed Thomsen-Friedenreich antigen in colon cancer is predominantly carried on high molecular weight splice variants of CD44. Glycobiology, 2001, 11(7), 587-592
Singh R., Subramanian S., Rhodes J. M., Campbell B. J., Peanut lectin stimulates proliferation of colon cancer cells by interaction with glycosylated CD44v6 isoforms and consequential activation of c-Met and MAPK: functional implications for disease-associated glycosylation changes. Glycobiology, 2006, 16(7), 594-601
Song S., Liu D., Peng J., Deng H., Guo Y., Xu L. X., Miller A. D., Xu Y., Novel peptide ligand directs liposomes toward EGF-R high-expressing cancer cells in vitro and in vivo. FASEB J, 2009, 23, 1396-1404
Tada H., Higuchi H., Wanatabe T. M., Ohuchi N., In vivo real-time tracking of single quantum dots conjugated with monoclonal anti-HER2 antibody in tumors of mice. Cancer Res., 2007, 67(3), 1138-1144
Torchilin V. P., Klibanov A. L., Immobilization of proteins on liposome surface. Enzyme Microb Tech, 1981, 3, 297-304
Torre L. G., Carneiro A. L., Rosada R. S., Silva C. L., Santana M. H. A., A mathematical model describing the kinetic of cationic liposome production from dried lipid films adsorbed in a multitubular system. Braz J Chem Eng, 2007, 24, 477-486
Withrow K. P., Gleysteen J. P., Safavy A., Skipper J., Desmond R. A., Zinn K., et al. Assessment of indocyanine green-labeled Cetuximab to detect xenografted head and neck cancer cell lines. Otolarvng Head Neck, 2007, 137, 729-734
Yaseen M. A., Yu J., Jung B. S., Wong M. S., Anvari B., Biodistribution of encapsulated Indocyanine Green in healthy mice. Mol Pharm, 2009, 6, 1321-1332
Yu J., Javier D., Yaseen M. A., Nitin N., Richards-Kortum R., Anvari B., Wong M. S., Self-assembly synthesis, tumor cell targeting, and photothermal capabilities of antibody-coated indocyanine green nanocapsules. J Am Chem Soc, 2010, 132, 1929-1938
Yuan B., Chen N., Zhu Q., Emission and absorption properties of indocyanine green in intralipid solution. J. Biomed. Opt., 2004, 9(3), 497-503

1. A particle comprising a water-insoluble polymer, wherein at least one near infrared (NIR) fluorescent probe is non-covalently bound to the outer surface of said particle.

2. The particle of claim 1, wherein said polymer is a cationic polymer.

3. The particle of claim 2, wherein said cationic polymer is an acrylic-, methacrylic-, or acrylic-methacrylic-copolymer such as Eudragit RS, Eudragit RS 30D, Eudragit RL 30D, Eudragit RL 100 and Eudragit RL PO, or chitosan.

4. The particle of claim 2, wherein said cationic polymer is admixed with a nonionic surfactant selected from the group consisting of a polysorbate such as Polysorbate 80 (Tween 80) and Polysorbate 20 (Tween 20), a hydrogenated castor oil such as Polyoxyl 40 hydrogenated castor oil (Cremophor RH 40), Polyoxyl 60 hydrogenated castor oil (Cremophor RH 60) and Polyoxyl 35 castor oil (Cremophor EL), a Pluronic block copolymer such as Pluronic L81, Pluronic P85, Pluronic F68, Pluronic F127 and Pluronic L44 NF INH, Lutrol F127, Solutol HS-15 or d-α-tocopheryl polyethylene glycol 1000 succinate (TPGS); and a surfactant such as lecithin and bile salts.

5. The particle of claim 2, further comprising a protein, peptide or polypeptide adsorbed to the outer surface of said cationic polymer.

6. The particle of claim 5, wherein said protein is casein, human serum albumin, bovine serum albumin, whey protein, β-lactoglobulin, α-lactalbumin, ovalbumin, lysozyme, a soy protein, or wheat protein.

7. The particle of claim 1, wherein said at least one fluorescent probe each independently is selected from the group consisting of a cyanine dye such as indocyanine green (ICG), Cy5, Cy5.5, Cy5.18, Cy7 and Cy7.18, IRDye 78, IRDye 680, IRDye 750, IRDye 800 phosphoramidite, DY-681, DY-731, DY-781, and an Alexa Fluor dye such as Alexa Fluor 610, Alexa Fluor 633, Alexa Fluor 647, Alexa Fluor 660, Alexa Fluor 680, Alexa Fluor 700 and Alexa Fluor 750.

8. The particle of claim 1, further comprising at least one active agent non-covalently bound to the outer surface of said particle.

9. The particle of claim 8, wherein said at least one active agent each independently is selected from the group consisting of a peptide, a polypeptide, a protein such as an antibody, a lectin, or a ligand-binding fragment thereof, a hormone or an analogue thereof, a glycoprotein, a lipoprotein, an amino acid, a polysaccharide, a glycolipid, a vitamin, a cofactor, a nucleoside, a nucleotide, and a nucleic acid such as RNA and DNA.

10. The particle of claim 9, wherein said at least one active agent is a targeting moiety capable of selectively binding the particle to a desired target.

11. The particle of claim 10, wherein:
(i) said target is a tumor such as a gastrointestinal cancer, or a pre-cancer state such as an adenoma or polyp, and said at least one active agent is capable of binding a specific cellular marker of said tumor or pre-cancer state such as a cell surface receptor or a cell surface glycoprotein;
(ii) said at least one active agent is an anti-epidermal growth factor receptor (EGFR) antibody such as cetuximab, an epidermal growth factor (EGF), or a lectin capable of binding galactosyl β-1,3-N-acetyl galactosamine (Gal β-1,3-GalNAc; Thomsen Friedenreich antigen) such as peanut agglutinin Arachis hypogaea lectin); or
(iii) a further active agent is non-covalently bound or embedded to said particle, and said further active agent is a therapeutic agent such as a chemotherapeutic agent.

12-13. (canceled)

14. A particle comprising a phospholipid, wherein at least one near infrared (NIR) fluorescent probe and at least one active agent are non-covalently bound to the outer surface of said particle.

15. The particle of claim 14, wherein:
(i) said phospholipid is selected from the group consisting of a lecithin such as egg or soybean lecithin; a phosphatidylcholine such as egg phosphatidylcholin; a hydrogenated phosphotidylcholine; a lysophosphatidylcholine; dipalmitoylphosphatidylcholine; distearoylphosphatidylcholine; dimyristoylphosphatidylcholine; dilauroylphosphatidylcholine; a glycerophospholipid such as phosphatidylglycerol, phosphatidylserine, phosphatidylethanolamine, lysophosphatidylethanolamine, phosphatidylinositol, phosphatidylinositol phosphate, phosphatidylinositol bisphosphate and phosphatidylinositol triphosphate; sphingomyelin; cardiolipin; a phosphatidic acid; a glycolipid such as a glyceroglycolipid, e.g., a galactolipid and a sulfolipid, a glycosphingolipid, e.g., a cerebroside (a glucocerebroside and a galactocerebroside), and a glycosylphosphatidylinositol; a plasmalogen; a phosphosphingolipid such as a ceramide phosphorylcholine, a ceramide phosphorylethanolamine and a ceramide phosphorylglycerol; and a mixture thereof;
(ii) said phospholipid is admixed with one or more nonphosphorous-containing molecules each independently selected from the group consisting of a fatty amine such as octylamine, laurylamine, N-tetradecylamine, hexadecylamine, stearylamine, oleylamine, tallowamine, hydrogenated tallowamine, and cocoamine; a fatty acid; a fatty acid amide; an ester of a fatty acid such as isopropyl myristate, hexadecyl stearate, and cetyl palmitate; cholesterol; a cholesterol ester; a diacylglycerol; and a glycerol ester such as glycerol ricinoleate; or
(iii) said phospholipid is admixed with one or more PEGylated phospholipids such as PEGylated dipalmitoyl phosphatidylethanolamine (DPPE-PEG), PEGylated palmitoyloleoyl phosphatidylethanolamine (POPE-PEG), PEGylated dioleoyl phosphatidylethanolamine (DOPE-PEG) and PEGylated distearoyl phosphatidylethanolamine (DSPE-PEG).

16-17. (canceled)

18. The particle of claim 15, comprising up to 15% by weight of 1,2-distearoyl-sn-glycero-3-phosphoethanolamine-N-[polyethyleneglycol 2000] (PEG-DSPE-2000).

19. The particle of claim 14, wherein:
(i) said at least one fluorescent probe each independently is selected from the group consisting of a cyanine dye such as indocyanine green (ICG), Cy5, Cy5.5, Cy5.18, Cy7 and Cy7.18, IRDye 78, IRDye 680, IRDye 750, IRDye 800 phosphoramidite, DY-681, DY-731, DY-781, and an Alexa Fluor dye such as Alexa Fluor 610, Alexa Fluor 633, Alexa Fluor 647, Alexa Fluor 660, Alexa Fluor 680, Alexa Fluor 700 and Alexa Fluor 750; or
(ii) said at least one active agent each independently is selected from the group consisting of a peptide, a polypeptide, a protein such as an antibody, a lectin, or a ligand-binding fragment thereof, a hormone or an analogue thereof, a glycoprotein, a lipoprotein, an amino acid, a polysaccharide, a glycolipid, a vitamin, a cofactor, a nucleoside, a nucleotide, and a nucleic acid such as RNA and DNA.

20. (canceled)

21. The particle of claim 19, wherein said at least one active agent is a targeting moiety capable of selectively binding the particle to a desired target.

22. The particle of claim 21, wherein:
(i) said target is a tumor such as a gastrointestinal cancer, or a pre-cancer state such as an adenoma or polyp, and said at least one active agent is capable of binding a specific cellular marker of said tumor or pre-cancer state such as a cell surface receptor or a cell surface glycoprotein;
(ii) said at least one active agent is an anti-epidermal growth factor receptor (EGFR) antibody such as cetuximab, an epidermal growth factor (EGF), or a lectin capable of binding galactosyl β-1,3-N-acetyl galactosamine (Gal β-1,3-GalNAc; Thomsen Friedenreich antigen) such as peanut agglutinin (Arachis hypogaea lectin); or
(iii) a further active agent is non-covalently bound or embedded to said particle, and said further active agent is a therapeutic agent such as a chemotherapeutic agent.

23-24. (canceled)

25. A powder comprising freeze dried- or spray dried-particles according to claim 1, optionally further comprising at least one cryoprotectant such as a sugar, a polymer, a protein, or an amino acid.

26. (canceled)

27. A pharmaceutical composition comprising particles according to claim 1, and a pharmaceutically acceptable carrier.

28. The pharmaceutical composition of claim 27, for oral administration, optionally:
(i) in the form of a monolithic matrix; a tablet; a capsule or sachet; or a depot system based on a biodegradable polymer such as poly(D,L-lactide) (PLA), polyglycolide (PGA), and poly(D,L-lactide-co-glycolide) (PLGA);
(ii) formulated for continuous sustained release, pulsatile release, or multi-phase release of the particles; or
(iii) further comprising an enteric coating.

29-35. (canceled)

36. A method for detection of a cancer or a pre-cancer state in the gastrointestinal tract of an individual in need, said method comprising administering to said individual a pharmaceutical composition according to claim 28, and detecting the presence of near infrared (NIR) emission from the walls of the gastrointestinal tract upon excitation at a proper wavelength, optionally wherein said particles comprise a water-insoluble polymer, to the outer surface of which a NIR fluorescent probe and a targeting moiety capable of selectively binding the particles to the tissue of said cancer or pre-cancer state are non-covalently bound.

37-39. (canceled)

40. A method for treatment of a cancer or a pre-cancer state in the gastrointestinal tract of an individual in need, said method comprising administering to said individual a therapeutically effective amount of a pharmaceutical composition according to claim 28, optionally wherein said particle comprises:
(i) a water-insoluble polymer, to the outer surface of which a NIR fluorescent probe, a targeting moiety capable of selectively binding the particles to the tissue of said cancer or pre-cancer state, and a chemotherapeutic agent are non-covalently bound; or
(ii) a water-insoluble polymer, to the outer surface of which a NIR fluorescent probe and a targeting moiety capable of selectively binding the particles to the tissue of said cancer or pre-cancer tissue are non-covalently bound, and a chemotherapeutic agent is non-covalently embedded to the particle.

41. (canceled)

42. A method for the preparation of an aqueous dispersion of particles comprising a water-insoluble polymer, to the outer surface of which at least one near infrared (NIR) fluorescent probe is non-covalently bound, said method comprising the steps of:
(i) dissolving a water-insoluble polymer in an organic solvent to obtain an organic solution of said polymer;
(ii) mixing said organic solution with an aqueous solvent in which a surfactant is optionally dissolved, in a ratio of 40:60 to 5:95, respectively, and removing said organic solvent to obtain an aqueous dispersion comprising particles of said polymer;
(iii) optionally mixing the aqueous dispersion of (ii) with an aqueous solution of a protein, peptide or polypeptide, to obtain an aqueous dispersion comprising particles of said polymer to the outer surface of which said protein, peptide or polypeptide is adsorbed;
(iv) mixing the aqueous dispersion of (ii) or (iii) with an aqueous solution of at least one NIR fluorescent probe thereby non-covalently binding said at least one fluorescent probe to the outer surface of said particles; and
(v) removing unbound molecules of said fluorescent probe from the dispersion obtained in (iv).

43. A method for the preparation of an aqueous dispersion of particles comprising a phospholipid, to the outer surface of which at least one near infrared (NIR) fluorescent probe and at least one active agent are non-covalently bound, said method comprising the steps of:
(i) providing an aqueous dispersion of particles comprising a phospholipid;
(ii) mixing the aqueous dispersion of (i) with an aqueous solution of at least one NIR fluorescent probe thereby non-covalently binding said at least one fluorescent probe to the outer surface of said particles;
(iii) removing unbound molecules of said fluorescent probe from the dispersion obtained in (ii);
(iv) mixing the aqueous dispersion of (iii) with an aqueous solution of said at least one active agent thereby non-covalently binding said at least one active agent to the outer surface of said particles; and
(v) removing unbound molecules of said at least one active agent from the dispersion obtained in (iv).

44. A powder comprising freeze dried- or spray dried-particles according to claim 14, optionally further comprising at least one cryoprotectant such as a sugar, a polymer, a protein, or an amino acid.

45. A pharmaceutical composition comprising particles according to claim 14, and a pharmaceutically acceptable carrier.

46. The pharmaceutical composition of claim 45, for oral administration, optionally:
(i) in the form of a monolithic matrix; a tablet; a capsule or sachet; or a depot system based on a biodegradable polymer such as poly(D,L-lactide) (PLA), polyglycolide (PGA), and poly(D,L-lactide-co-glycolide) (PLGA);
(ii) formulated for continuous sustained release, pulsatile release, or multi-phase release of the particles; or
(iii) further comprising an enteric coating.

47. A method for detection of a cancer or a pre-cancer state in the gastrointestinal tract of an individual in need, said method comprising administering to said individual a pharmaceutical composition according to claim 46, and detecting the presence of near infrared (NIR) emission from the walls of the gastrointestinal tract upon excitation at a proper wavelength, optionally wherein said particles comprise a phospho lipid, to the outer surface of which a NIR fluorescent probe and a targeting moiety capable of selectively binding the particles to the tissue of said cancer or pre-cancer state are non-covalently bound.

48. A method for treatment of a cancer or a pre-cancer state in the gastrointestinal tract of an individual in need, said method comprising administering to said individual a therapeutically effective amount of a pharmaceutical composition according to claim 46, optionally wherein said particles comprise:
(i) a phospholipid, to the outer surface of which a NIR fluorescent probe, a targeting moiety capable of selectively binding the particles to the tissue of said cancer or pre-cancer state, and a chemotherapeutic agent are non-covalently bound; or
(ii) a phospho lipid, to the outer surface of which a NIR fluorescent probe and a targeting moiety capable of selectively binding the particles to the tissue of said cancer or pre-cancer tissue are non-covalently bound, and a chemotherapeutic agent is non-covalently embedded to the particle.
